(19) United States
(12) Patent Application Publication
(10) Pub. No.: US 2026/0144468 A1
(43) Pub. Date: May 28, 2026

(54) TECHNIQUES TO AUTOMATICALLY DETECT REGIMES OF EXPLORATORY NEUROMOTOR LEARNING IN A SUBJECT

(71) Applicant: RUTGERS, THE STATE UNIVERSITY OF NEW JERSEY, New Brunswick, NJ (US)

(72) Inventor: Elizabeth B. TORRES, Piscataway, NJ (US)

(21) Appl. No.: 19/123,472

(22) PCT Filed: Oct. 26, 2023
(86) PCT No.: PCT/US2023/077831

§ 371 Date: Apr. 23, 2025

Related U.S. Application Data
(60) Provisional application No. 63/381,199, filed on Oct. 27, 2022.

(51) Int. Cl. A61B 5/16; G06N 20/00

(52) U.S. Cl. CPC A61B 5/16; G06N 20/00

(57) ABSTRACT

Techniques for distinguishing exploratory learning from error-based learning in a subject includes presenting sensory stimuli between a start time and an end time divided into multiple time blocks of equal duration. A Gamma function (Γ) scale and shape parameter value pair that fit micropeak statistics within sensor data is determined during each of one or more windows of equal duration in each block. A profile of Γ scale and shape parameter value pairs is determined in shape-scale space during each block. The subject is determined to be engaged in exploratory learning during each block in which variance in that block exceeds a threshold variance based on a Γ scale and shape parameter value pair selected along the profile. The detection of exploratory learning distinguishes neurodevelopment among subjects and provides alternative training approaches for machine learning.

### CROSS-REFERENCE TO RELATED APPLICATIONS

This application claims benefit of Provisional ApplNo. 63/381,199, filed Oct. 27, 2022, the entire contents of which are hereby incorporated by reference as if fully set forth herein, under 35 U.S.C. § 119 (c).

### BACKGROUND

In previous work, inventors have shown that neural activity is reflected in micromovements of a subject. Micromovements are defined as subtle motions that rise and fall on time scales less than one second. These micromovements, and associated neural activity, are characterized by micropeaks in signals from movement sensors or neural sensors, where a micropeak is a peak in the signal that rises and falls in less than one second. The statistics of micropeak features (features including, for example, micropeak frequency, time between successive micropeaks, micropeak amplitude, micropeak prominence, micropeak width, or normalized versions of same, or cross-correlations among micropeaks in different signals, among others) have been used in previous work to distinguish intentional activity from subconscious activity by the subject and to distinguish differences in neuromotor development, for example, differences between subjects on the Autism Disorder Spectrum (ADS) and normally developing subjects. In various implementations, signals from an electroencephalogram (EEG) of a subject, auditory brainstem response (ABR) sensors, acoustic sensors, accelerometers attached to a subject, and video data observing the subject have been used to detect micropeaks and statistics of micropeak features. Such previous work is published in patent publications US20140336539, US20170344706, US20170340261, US20190254533, US20190261909, US20190333629, and pending international publications of PCT applications PCT/US2023/67728 and PCT/US2023/67729, the entire contents of each of which are hereby incorporated by reference as if fully set forth herein.

Statistics of a micropeak feature are defined by that feature's empirical probability density function (pdf). It has been found that such empirical pdfs are usefully fit by the Gamma function (Γ) by adjusting values of Γ parameters shape and scale. Patterns in the variation of the empirically determined shape and scale values are then used to characterize or distinguish neuromotor development of one or more subjects. An asserted advantage of the empirical pdf approach using micropeaks is to free the methods from an artificial imposition of standard or convenient, or analytically simple, pdfs, such as pure Gaussian distributions. Because the statistics are based on micropeaks, it is practical to obtain sufficient data to define pdfs empirically on a relatively short time scale compared to the duration of neuromotor tasks being evaluated.

### SUMMARY

Techniques are provided for using empirically determined micropeak statistics to distinguish learning approaches during sessions of predictable, unpredictable and mixed stimuli that are not previously known to a subject. This approach enables both distinguishing types of learners among human subjects, identifying scenarios in which different learning approaches are efficacious, determining neurodevelopment of a subject based on different learning types, and alternative training approaches for machine learning.

In a first set of embodiments, a method implemented on a processor for distinguishing exploratory learning from error-based learning in a subject includes presenting, to a subject, sensory stimuli beginning at a start time and ending at an end time. A stimuli time difference between the start time and the end time is more than 10 minutes and less than 12 hours and is divided into multiple time blocks of equal duration. The method also includes determining a Gamma function (Γ) scale and shape parameter value pair that fit micropeak statistics within sensor data observing the subject during each window of one or more windows of equal duration in each time block. Each window has a duration such that an average number of micropeaks in the windows in the block is at least 100 micropeaks. The method further includes determining a profile of Γ scale and shape parameter value pairs in shape-scale space during each block. Even further still, the method includes determining that the subject is engaged in exploratory learning during each block in which variance in that block exceeds a threshold variance based on a Γ scale and shape parameter value pair selected along the profile.

In some embodiments of the first set, the sensory stimuli include predictable and unpredictable changes. In some embodiments of the first set, the sensory stimuli comprise visual-motor stimuli.

In some embodiments of the first set, the sensor data is collected at a rate greater than 100 Hertz (Hz), or greater than 150 Hz, or greater than 200 Hz, or greater than 250 Hertz, or greater than 300 Hz.

In some embodiments of the first set, each window has a duration selected in a range from one second to 10 seconds. In some embodiments of the first set, each window overlaps in time any window adjacent in time. In some of these embodiments, each window overlaps any window adjacent in time by 50%. In some embodiments of the first set, each block includes at least 20 windows.

In some embodiments of the first set, the method includes determining distance between distributions in successive windows, wherein distance is Earth Mover's distance. In some embodiments of the first set, the threshold for exploratory learning is a percentile of Γ scale values along the profile. In some of these embodiments, the percentile is the 50^{th} percentile.

In some embodiments of the first set, the method even further yet includes determining a neurological condition of the subject based upon a percentage of time during the presentation of the sensory stimuli during in which the subject is engaged in exploratory learning.

In a second set of embodiments, a method implemented on a processor for adjusting values of parameters for a machine learning system includes adjusting parameter values for the machine learning algorithm using exploratory learning. Exploratory learning comprises a sequence of one or more probability density functions used by a subject engaged in a block of exploratory learning determined by the following steps. Determining exploratory learning includes presenting, to a subject, sensory stimuli beginning at a start time and ending at an end time. A stimuli time difference between the start time and the end time is more than 10 minutes and less than 12 hours and is divided into multiple time blocks of equal duration. Determining exploratory learning also includes determining a Gamma function (Γ) scale and shape parameter value pair for micropeak statistics within sensor data observing said subject during each window of one or more windows of equal duration in each time block. Each window has a duration such that each window has on average at least 100 micropeaks. Determining exploratory learning further includes determining a profile of Γ scale and shape parameter value pairs during each block. Even further still, determining exploratory learning includes determining that the subject is engaged in exploratory learning during each block in which variance in that block exceeds a threshold variance based on a Γ scale and shape parameter value pair selected along the profile.

In other sets of embodiments a computer-readable medium or an apparatus is configured to cause an apparatus to perform the steps of one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example of a system for detecting exploratory learning, according to an embodiment;

FIG. 2A through FIG. 2G are plots that illustrate examples of micropeaks and empirical micropeak statistics in data obtained from the system of FIG. 1, according to various embodiments;

FIG. 3A is a block diagram that illustrates an example of a distance metric between distributions, such as empirical probability density functions of micropeak statistics, used in some embodiments;

FIG. 3B and FIG. 3C are plots that illustrate example traces of distance between micropeak statistics in successive windows of time within one block of data, according to an embodiment;

FIG. 4A through FIG. 4C are plots the illustrate examples of statistics of distances between successive windows across all blocks, according to an embodiment;

FIG. 5 is a plot that illustrates examples of differences in variance of pdfs among blocks that exhibit two types of learning, according to an embodiment;

FIG. 6 is a flow chart that illustrates an example of a method to detect and use exploratory learning; according to an embodiment;

FIG. 7A is a block diagram that illustrates a schematic relationship among processes in a learning session, according to an experimental embodiment;

FIG. 7B through FIG. 7F are plots that illustrate examples of micropeak distributions during experimental learning sessions by one or more subjects, according to an embodiment;

FIG. 8A through FIG. 8F are plots that illustrate examples of differences between micropeak distributions during different experimental learning sessions by one or more subjects, according to an embodiment;

FIG. 9A through FIG. 9C are plots that illustrate examples of temporal evolutions of variances in micropeak distributions during different experimental learning sessions by one or more subjects, according to an embodiment;

FIG. 10A and FIG. 10B are plots that illustrate scores of subjects engaged in experimental learning sessions relative to their evolutions of micropeak variances, according to an embodiment;

FIG. 11A through FIG. 11C are plots that illustrate temporal evolution of Gamma function (Γ) statistics of micropeak distributions during different experimental learning sessions by one or more subjects, according to an embodiment;

FIG. 12A through FIG. 12F are plots that illustrate temporal evolution of Gamma function (Γ) parameters of micropeak distributions for different shapes and different learning types during experimental learning sessions by one or more subjects, according to an embodiment;

FIG. 13A through FIG. 13D are plots that illustrate distributions of distances between successive micropeak distributions during experimental learning sessions by one or more subjects, according to an embodiment;

FIG. 14 is a block diagram that illustrates a computer system upon which an embodiment of the invention may be implemented;

FIG. 15 illustrates a chip set upon which an embodiment of the invention may be implemented; and

FIG. 16 is a diagram of exemplary components of a mobile terminal (e.g., cell phone handset) for communications, which is capable of operating in the system, according to one embodiment.

### DETAILED DESCRIPTION

A method and apparatus are described for detecting and using exploratory learning. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

### 1. OVERVIEW

Some embodiments of the invention are described below in the context of micropeak statistics during different types of learning of a visual-motor task by normal adults. However, the invention is not limited to this context. In other embodiments learning statistics differences are detected for auditory learning, motor learning, and other sensory learning, alone or in some combination, by neurologically normal and neurologically atypical subjects, whether adult, immature or neonate. In some embodiments, the learning statistics are used to distinguish types of learning, to distinguish scenarios for which different types of learning are effective, to distinguish normal from atypical neurological conditions, or as a basis for machine learning, or some combination.

### 1.1 System Structures

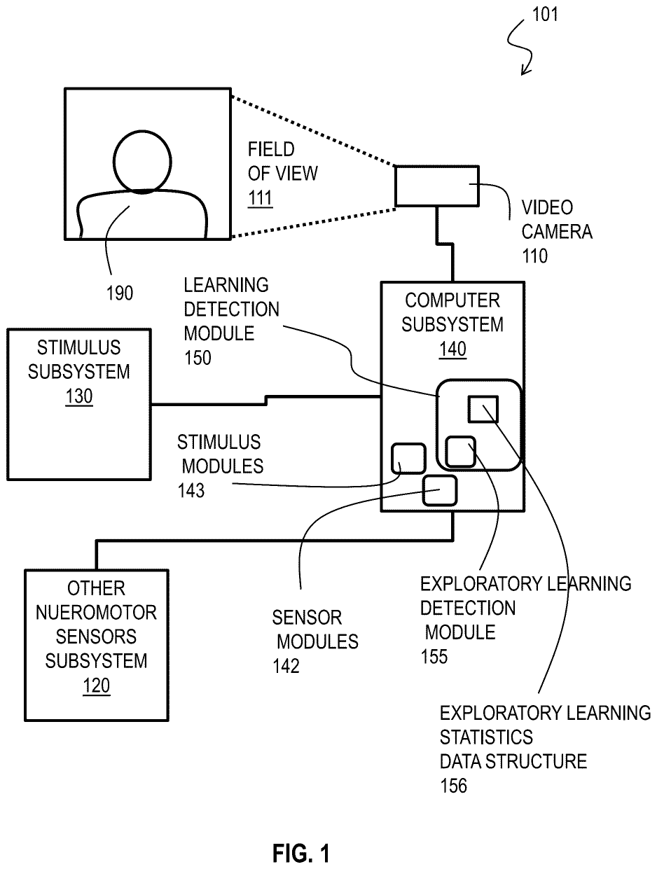

FIG. 1 is a block diagram that illustrates an example of a system 101 for detecting exploratory learning, according to an embodiment. System 101 includes a video camera 110 configured to capture and record digital video data of objects in a field of view 111, such objects including one or more subjects, such as subject 190. Although subject 190 is included in FIG. 1 for purposes of illustration, subject 190 is not part of system 101. System 101 also includes other neuromotor sensors subsystem 120, such as movements sensors, like orientation sensors or accelerometers, and electrical sensors such as electroencephalogram (EEG), electrocardiogram (ECG), and auditory brainstem response (ABR) electrodes, acoustic sensors such as echocardiogram (EKG) transducers, and optical sensors such as pulse oximeter transceivers, attached to various parts of the subject 190. The system also includes a stimulus subsystem 130 configured to present sensory stimuli to the subject 190. Sensory stimuli may include visual stimuli, pressure or other tactile forces, sounds, chemical dispensers for taste or smell stimuli, heating or cooling mechanisms to produce temperature stimuli, and so forth, alone or in some combination in various embodiments, using any stimuli presentation methods known.

The computer subsystem 140 includes one or more sensor modules 142 configured to detect the spatial arrangement or movement of a variety of anatomical joints of subject 190 in each frame of the video data, e.g., using pose detection software, as well as output from any of the other sensors in subsystem 120. The computer subsystem 140 includes one or more stimulus modules 143 configured to operate the stimulus subsystem 130. The computer subsystem 140 includes a learning detection module 150 configured to infer the types of learning by a subject captured in the video or other sensor data based on distributions of sub-second peaks, also sometimes called micromovement spikes (MMS) or micro-peaks herein, in each of one or more parts of the subject 190 as described in more detail below. As used herein, a micromovement is a signal change accomplished on the time scales of tens of microseconds to hundreds of milliseconds whether the signal reflects physical motion or electrical variations. In some embodiment, the learning detection module 150 includes an exploratory learning module 155 and one or more data structures, such as exploratory learning statistics data structure 155.

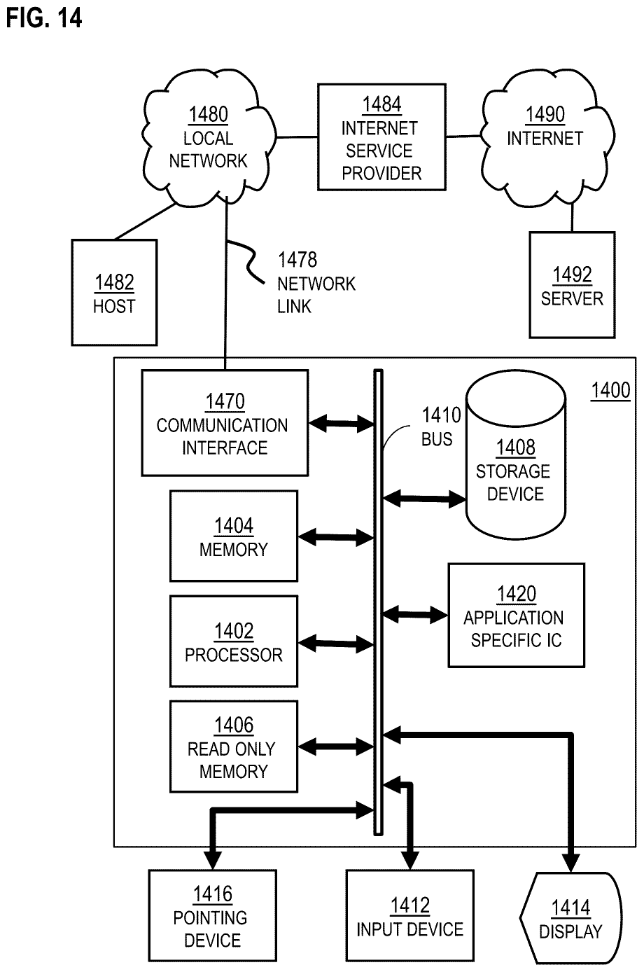

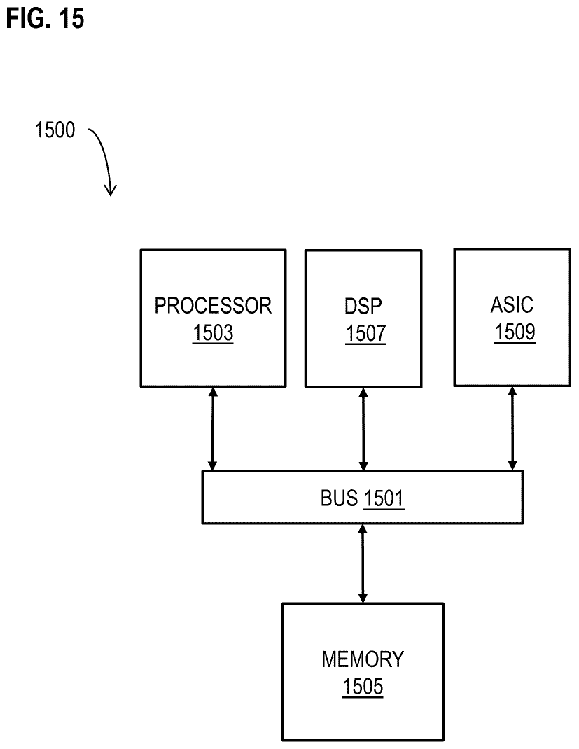

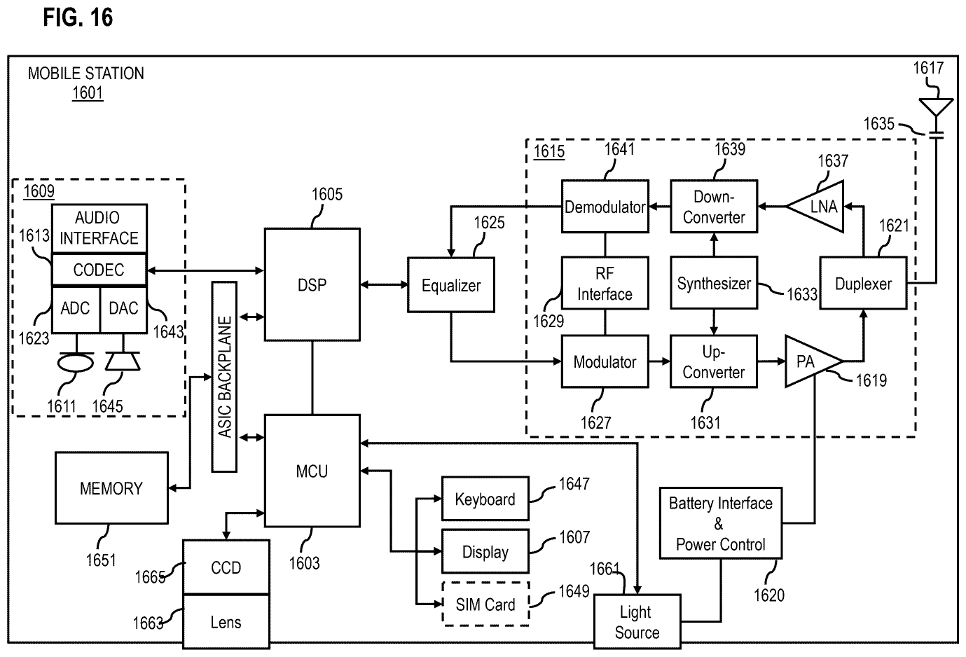

In some embodiments, the computer subsystem 140 includes one or more computer systems or hosts, such as computer system 1400 described in more detail below with reference to FIG. 14. In some embodiments, the computer subsystem 140 includes one or more chip sets in one or more special devices, such as chip set 1500 described in more detail below with reference to FIG. 15. In some embodiments, the computer subsystem 140 includes one or more mobile communication terminals, such as mobile terminal 16 described in more detail below with reference to FIG. 16. In some of these embodiments, the video camera 110 is included within the mobile terminal 1600, such as in a modern smart cell phone, including lens 1663, charge coupled device 1665 and light source 1661 as described in more detail below.

In some embodiments, the learning detection module 150 includes one or more other modules, such as exploratory learning module 155 and one or more data files or databases (e.g., exploratory learning statistics data structure 156) that hold data that indicate one or more predetermined distributions of micropeaks or changes thereof, or both, of learning types and timing in one or more populations of control individuals, each population associated with a different neurodevelopmental condition, such as a diagnosis of ASD or no ASD (typically developing, TD).

Any known algorithm or software application can be used as the video pose component of sensor modules 142. Several different algorithms for pose estimation have been published over the past decade (e.g., OpenPose (Cao et al., 2017), DeepLabCut (Mathis et al., 2018), DeepPose (Toshev et al., 2014), DeeperCut (Insafutdinov et al., 2016), AlphaPose (Fang et al., 2018), ArtTrack (Insafutdinov et al., 2017)). Using these algorithms, it is possible to take advantage of pretrained networks that are freely available, or train new networks customized for various research or clinical needs. For example, a commonly used pretrained network is the human pretrained demo of OpenPose that includes key points of the body, feet, hands, and face and has been used in several recent studies for quantitative analysis of human movement, including VideoPose.

Although processes, equipment, and data structures are depicted in FIG. 1 as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts. For example,

### 1.2 Micropeaks in Sensor Data

The next component of the technique is to determine the trajectories of motion or electrical signals of any measured part of subject 190 using video camera 110 or other neuromotor sensors subsystem 120.

Video data is converted to scalar motion. For example, scalar speed is pixel distance, d_{P}, per time for one video frame, where d_{P} is given by Equation 1.

$${d}_{P} ( i ) = \sqrt{{( {X}_{i} - {X}_{i - 1} )}^{2} + {( {Y}_{i} - {Y}_{i - 1} )}^{2}} \qquad ( 1 )$$

There are micromovement spikes (MMS) in scalar speed profiles for many joints or other portions of the subject 190. In other embodiments, other parameters of micro movement can be used, such as scalar acceleration, X-speed, Y-speed, X-acceleration, Y-acceleration, among others.

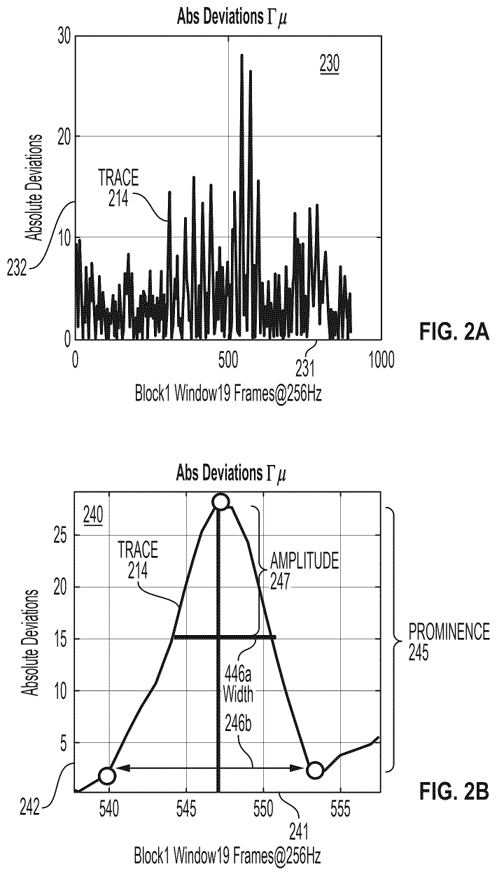

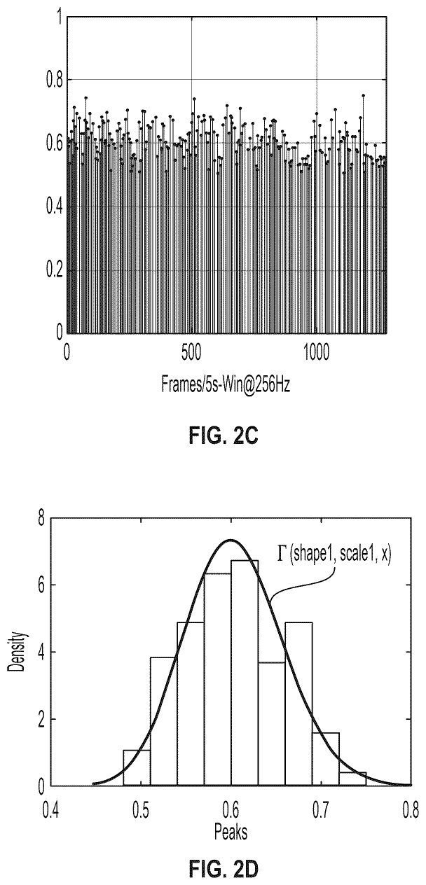

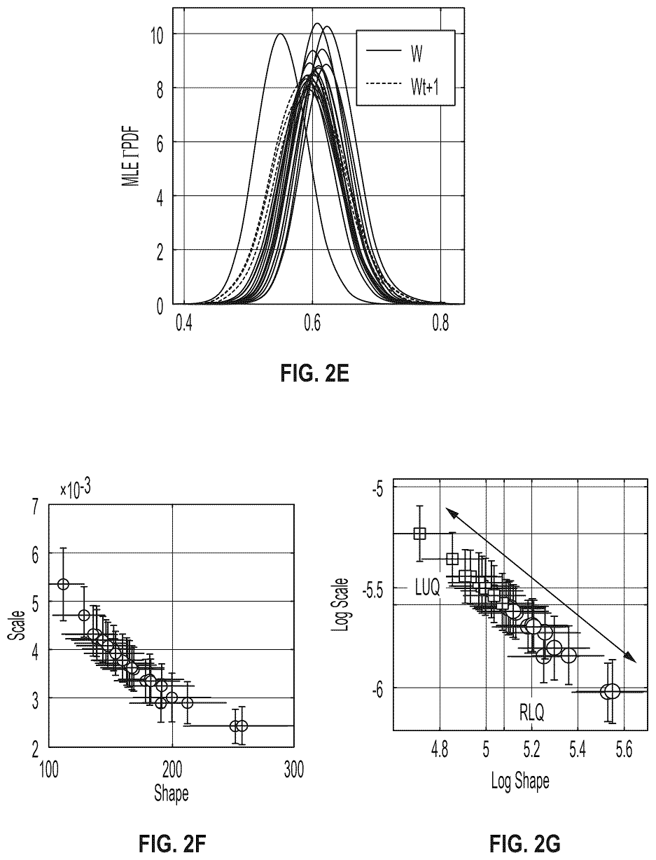

FIG. 2A through FIG. 2G are plots that illustrate examples of micropeaks and empirical micropeak statistics in data obtained from the system of FIG. 1, according to various embodiments. For each graph of FIG. 2A and FIG. 2B, the horizontal axis 231 or 241 indicates a number of frames of EEG data taken at 256 Hertz (samples per second) so less than 1000 frames indicates a duration of under 4 seconds. The vertical axis 232 or 242 indicates deviations of an amplitude of a signal from a mean value, such as scalar speed or scalar acceleration, or EEG voltage among others, in arbitrary units. Trace 214 includes multiple fluctuations. A micropeak is considered to be the maximum amplitude deviation value between successive minimum amplitude deviation values. FIG. 2A shows in plot 230 multiple micropeaks along trace 214. FIG. 2B shows in plot 240 a single micropeak from FIG. 2A. The single micropeak in FIG. 2B at frame 547 along the horizontal axis has properties that include a prominence 245 from the largest minimum value on each side of the peak to the maximum value, an amplitude 247 of half the prominence value, a width 246a at half height, and a width 246b at the base which is at the value of the maximum of the two minima bracketing the micropeak.

In some embodiments, the micropeaks in the profile are normalized by the value of the largest prominence of all the micropeaks in the data being processed. In some embodiments, the normalization of any micropeak is computed instead using Equation 2.

$$P {n}^{*} = Pn / [ Pn + Avg ( P min 1 , P min 2 ) ] \qquad ( 2 )$$

Where Pn is the prominence of the nth micropeak, Pn* is the normalized prominence of the nth micropeak, and Avg(Pmin1, Pmin2) is the average of the two local minima Pmin1 and Pmin2, including the local maximum Pn When the Avg(Pmin1, Pmin2) is small compared to the prominence of the nth micropeak, the value of Pn* approaches 1. When the Avg (Pmin1, Pmin2) is large compared to the prominence of the nth micropeak, the value of Pn* approaches 0. Such normalization tends to scale out allometric differences in length, weight, anatomical size, and other characteristics for individuals in the same group (e.g., same age or stage of development or neurodevelopmental condition or combination).

FIG. 2C is a graph that illustrates example normalized micropeaks in an example EEG measurement. The horizontal axis indicates frame, and the vertical axis indicates normalized prominences of micropeaks. Each micropeak is plotted at the frame where it occurs with a normalized prominence value between 0 and 1. In the illustrated plot of FIG. 2D, measurement frames are taken at 256 Hertz (samples per second) for a sampling window of 5 seconds, so there are 1,280 samples in the window, as indicated by the horizontal axis value. In these 1,280 samples there are about 100 normalized micropeaks, depicted in FIG. 2C as vertical bars topped by a dot, in a range from about 0.5 to 1.0.

FIG. 2D is an example empirical histogram that indicates the number of micropeaks on the vertical axis for each normalized micropeak prominence value on the horizontal axis, binned in intervals of 0.025. This histogram represents an empirical probability distribution function (pdf) of micropeaks statistics (also called teh stochastic signature) during the sampling duration (e.g., the 1,280 frames in the window of FIG. 2C). Such empirical histograms can be characterized by the continuous Gamma Function (Γ) by adjusting values of the two Γ parameters (shape and scale) until a best fit is obtained to the data. An example Γ fit to the empirical histogram using maximum likelihood estimation (MLE) gives values shapel and scalel for the shape and scale parameters, respectively, and plotted as a function of the horizontal axis value x, representing the normalized prominence of the micropeaks, such as those in FIG. 2C, and represented as a solid line trace in FIG. 2D. In other embodiments, other fitting optimization measures known in the art are used, such as least squares, maximum entropy, etc.

During an experiment the sampling time forms a block that is divided into a number of windows of equal duration. The windows may overlap, or follow one another with no gaps, or be separated by gaps. The statistics of micropeaks in each window are determined separately and the Γ is fit to each window. By definition, the area under each Γ is the same, equal to one. The Γ fits for all the windows in one block are plotted together in FIG. 2E. As can be seen in FIG. 2E, the pdfs form a variety of different empirical curves. For each empirical curve, defined by a pair of shape and scale values, the moments of the distribution can be calculated as functions of the shape and scale parameter values, a and b, respectively. Such moments include the mean (Γμ), the variance (Γσ), the Γ′skewness and the Γkurtosis, among other moments. The first four moments are defined by Equation 3a through Equation 3d.

$$Γ μ = a \cdot b \qquad ( 3 a )$$

$$Γσ = a \cdot {b}^{2} \qquad ( 3 b )$$

$$Γskewness = \frac{2}{\sqrt{a}} \qquad ( 3 c )$$

$$Γkurtosis = 3 + \frac{6}{a} \qquad ( 3 d )$$

The relevance of such a variety of curves and moments to different types of learning is explained in more detail below.

FIG. 2F is a graph that illustrates example Γ parameters values for distributions of normalized micropeaks in one or more blocks. The horizontal axis indicates values for the Γ shape parameter (a) and the vertical axis indicates values for the Γ scale parameter (b). Points are plotted for about 20 window fits (55 seconds duration) in one block with 95% error bars. Teh 95% confidence bars are usefully small if a window size is selected such that an average number of micropeaks in the window is about 100 or more, such as in a range from 100 to 500. For the EEG data, a window size of 5 seconds satisfies this target. It has been noted before that a typical range of shape and scale parameters in neurodevelopment scenarios often lie along a relatively straight-line segment in a log-log plot. FIG. 2G is a graph that plots the points and error bars of FIG. 2F with the horizontal axis indicating the log of values for the Γ shape parameter and the vertical axis indicating the log of values for the Γ scale parameter. This kind of plot is called a log-log shape-scale plot, herein, and the space plotted the log-log shape-scale plane. The points do seem to lie along a straight-line segment.

This characterization has reduced the parameters of interest to one (the shape or the scale) since knowing one, one can infer the other with high certainty. Focusing below then on the ranges of pdf shapes, one can track the stochastic signature evolution during a learning session. The Γ parameter values reflect different degrees of variation and different levels of persistence in the neuromotor activity of the subject during learning. If the persistent part of the activity, at least over the local time scale of one window, the Γ mean (Γμ) is called the signal and the random fluctuations the Γ variance (Γσ), sometimes called the noise in inventor's own publications, at least over the local time scale, then the local noise to signal ratio (NSR) can be determined. But for clarity, we will use the term variance to mean ratio (VMR) hereinafter when the variance is a property of the stochastic process. Let a indicate the value of the Γ shape parameter and b a value of the Γ scale parameter. Then VMR is given by Equation 4.

$$V M R = \frac{Γ 0}{Γ μ} = \frac{a \cdot {b}^{2}}{a \cdot b} = b \qquad ( 4 )$$

A point (shape-scale values pair) at the median value of the shape values and the median value of the scale values is used as a center point for the block of windows; and the center point divides the other empirical value pairs into those that fall into a left upper quadrant (LUQ) plotted as open square symbols, and a right lower quadrant (RLQ) plotted as open circles.

The difference between the most extreme distributions of windows in the block is represented by the double pointed arrow in FIG. 2G. This difference can be measured by the vector difference between the shape-scale points in log-log space, such as the Euclidean difference (square root of sum of squares of differences in the coordinates of the two points) or the signed difference of either of the two coordinates, or the absolute value of the larger of the two differences in coordinates, among others, or, because of the linear relationship between shape and scale, by either shape or scale differences separately. Various other metrics for the difference between two distributions can be used.

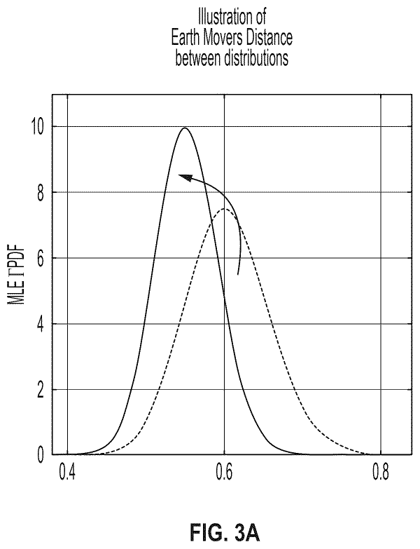

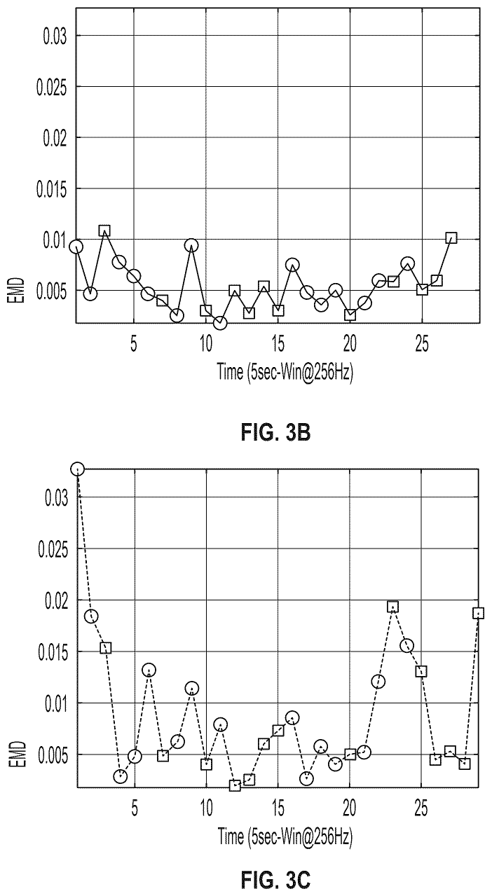

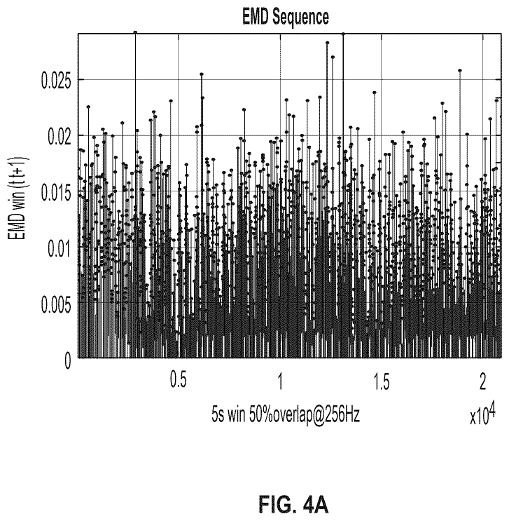

FIG. 3A is a block diagram that illustrates an example of a distance metric between distributions, such as empirical probability density functions of micropeak statistics, used in some embodiments. The Earth Movers Distance (EMD) offers an advantage of providing a distance-like measure of dissimilarity between two frequency distributions. FIG. 3A shows two distributions, represented by solid and dashed line curves, of equal area under the curves. The EMD is a computation of the work required to move areas that are inside the dashed curve and outside the solid into a previously unoccupied spot that is inside the solid curve but outside the dashed curve, i.e., the work required to change the dashed curve to the solid curve. The concept of the EMD computation is illustrated by the arrow in FIG. 3A. The more similar the two curves are, the less work is required and the smaller is the EMD. The more dissimilar the curves are, the more work is required and the larger is the EMD.

FIG. 3B and FIG. 3C are plots that each illustrate an example trace of EMD between micropeak statistics in successive windows of time within one block of data, according to an embodiment. In each plot, the horizontal axis is time expressed as window number (each window representing 5 seconds in this example); and, the vertical axis is EMD between the distributions of successive windows in time. The quadrant of FIG. 2F in which the distribution winds up after each change is indicated by an open square for left upper quadrant (LUQ) and by an open circle for the right lower quadrant (RLQ). FIG. 3B represents a block in which a subject is not changing micropeak statistics very much from one window to the next, with EMD values at or below about 0.01. For various reasons described in more detail below, such a block of low EMD signal is considered characteristic of error-based learning in which a subject has a model or target pattern from memory in mind that the subject is trying to fit to the current situation. FIG. 3C represents a block in which a subject is changing micropeak statistics much more from one window to the next, with some EMD values at or above about 0.02. For various reasons described in more detail below, such a block of high EMD signal is considered characteristic of exploratory learning in which the subject does not have a memory of a target pattern in mind and is reacting with wonder, awe or surprise at seeing a potential or candidate pattern in the here and now.

### 1.3 Peaks in Distribution Changes

The EMD profiles depicted in FIGS. 3B and 3C, or other scalar measures of the distribution in each window, or changes between successive windows, can also be treated as signals that can be characterized by peaks (e.g., multi-second EMD peaks as distinguished from micropeaks in a movement or electrical signal depicted in FIG. 2A) and the distribution of such measures can be characterized by Γ mean and variances deduced from Γ shape and scale parameter values found by fitting the observed multi-second variations.

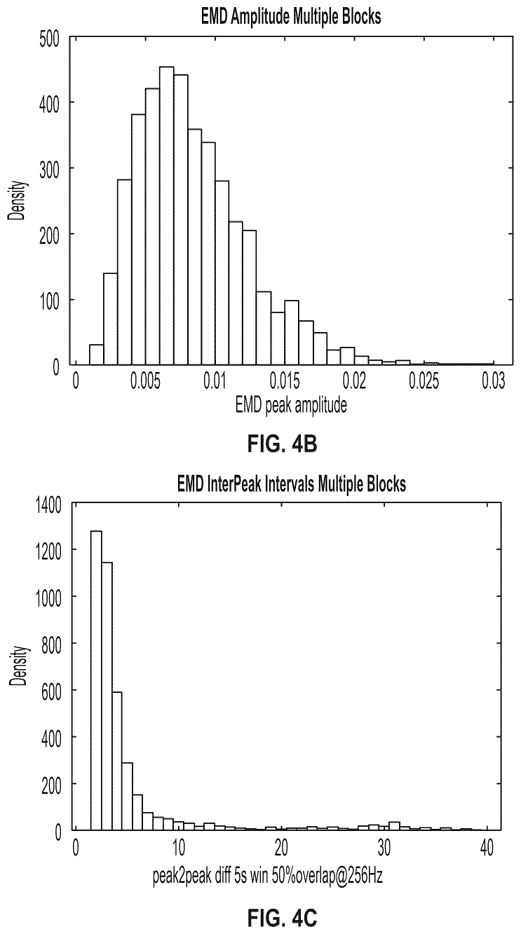

FIG. 4A through FIG. 4C are plots that illustrate examples of statistics of distances between successive windows across all blocks, according to an embodiment. FIG. 4A shows peaks in EMD changes between successive windows (at times t and t+1) for over 20,000 windows in an example experiment. A few hundred EMD peaks are evident in the signal. An amplitude or normalized prominence can be computed for each peak and the distribution of such amplitudes is plotted in FIG. 4B. Here the EMD peak amplitude value is indicated along the horizontal axis and the density (peak count per bin) is indicated along the vertical axis. The mean EMD peak is about 0.01 with a long tail at larger amplitudes. A time interval between peaks can be computed for each successive pair of peaks and the distribution of such interpeak intervals is plotted in FIG. 4C. Here the EMD interpeak interval value is indicated along the horizontal axis expressed in number of 5-second windows, spaced 2.5 seconds apart; and the density (interval count per bin) is indicated along the vertical axis. The mean EMD interpeak interval is about 5 windows (12.5 seconds) with a short tail at larger intervals. Either or both distributions can be fit with a Γ to derive shape and scale of EMD peaks and the moments of the distribution based on those shape and scale values.

1.4 Learning Associated with Variance in Micropeak Statistics

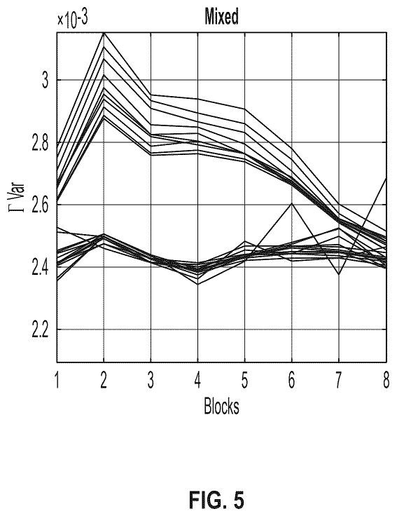

FIG. 5 is a plot that illustrates examples of differences in variance of profiles among blocks that exhibit two types of learning, according to an embodiment. The variance for a block refers to the variance combining all windows in one block of some measure of the distribution in each window—or a change thereof, e.g., the shape value for each window or the shape value change between successive windows. In the illustrated embodiment, the variance for each block is variance derived from the Γ fit for all in the block. Here the variance indicates the variance derived from the Γ fit and so is labeled in the plot Γ Var The horizontal axis indicates time in blocks. In this example plot, each block represents about twenty contiguous non-overlapping 5-second windows (100 seconds, or 40 50% overlapping 5-second windows) and 8 blocks represents 800 seconds (13 minutes 20 seconds). The vertical axis indicates variance from the Γ fit to micropeaks for each block in arbitrary units.

Each plotted profile represents a different subject. All subjects were exposed to the same learning task, as described in more detail below for an example embodiment. In general, that learning task involved identifying one of two figures in a sequence of noisy images. Sometimes the figures repeat in the sequence in a discernible pattern and sometimes the figures changed at random, thus producing a so-called “mixed” learning session. In other embodiments other learning tasks with repeating components and noise are presented to the subject by the sensory stimulus modules 143.

The subjects' profiles plotted in FIG. 5 clustered into two types. One type (type A) started at early blocks (low block number) with higher block variance and eventually settled on a lower block variance at the later blocks (high block number). Another type (type B) maintained a near constant block variance throughout the session of 8 blocks . . .

As this data shows, each subject in both types start naïve, without empirical knowledge or memory of the stochastic process at hand. This analysis does not make theoretical assumptions about this process (e.g., that it is Gaussian, stationary, linear, etc.). Instead, for short times, window by window, empirical stochastic signatures of data parameters (e.g., signals' normalized prominence and timing) determined and tracked how they evolve over time, as the learning unfolds. At the global level, whole block by whole block for the learning session, the fluctuations in those empirical stochastic signatures are examined a posteriori to gain insight into the overall dynamics of the statistical learning process that took place. For example, the evolution of the empirically estimated variance, as depicted in FIG. 5, were tracked.

Generally, at the leftmost extreme of a shape-scale plot like FIG. 2G, when the Gamma shape is 1, one has the special case of the memoryless exponential distribution (no points appear in this range for this example). This is the case of having a random process whereby events in the past do not inform more about events in the future than current events would. All future events are equally probable. The information is coming from the here and now. At this level of randomness, prior research has shown corresponding highest levels of VMR (We note that the signal mean to variance ratio MVR=1/VMR and is also used herein, and sometimes called signal to noise ratio, SNR, in inventor's own prior publications). Such distributions are typical for the motor code at the start of neurodevelopment. Around 4-5 years of age, when the subject is (on average) mature enough to start schooling, receive instructions, and sustain longer attention spans, a transition into heavy tailed distributions is observed. By college age these distributions are tending to Gaussian, so that the shape parameter is at the other extreme of the shape axis on the Γ parameter plane and the SNR is at its highest value (FIG. 2G). Prior work has also revealed that in subjects where maturation is compromised (e.g., autism across the lifespan) these global signatures remain in the exponential range, randomly relying on the here and now and manifesting very low SNR. In this case, the system does not progress into acquiring a predictive code.

As used herein, exploratory learning is associated with large variance of low-mean-large-tailed distributions (LUQ), which is closer to memoryless exponential distributions (all future events equally probable) than other empirical points on the shape-scale profile. This type of learning is ascribed to the high Γ Var subjects in block 1 and continuing at higher Γ Var through block 7. In contrast error-correction learning is associated with small variance tending toward the RLQ. This type of learning is ascribed to the low Γ Var subjects in block 1 and continuing through block 8. In blocks 7 and 8 the exploratory learning subjects have evolved to approach steady use of error-correction learning.

1.5 Method to detect and use exploratory learning

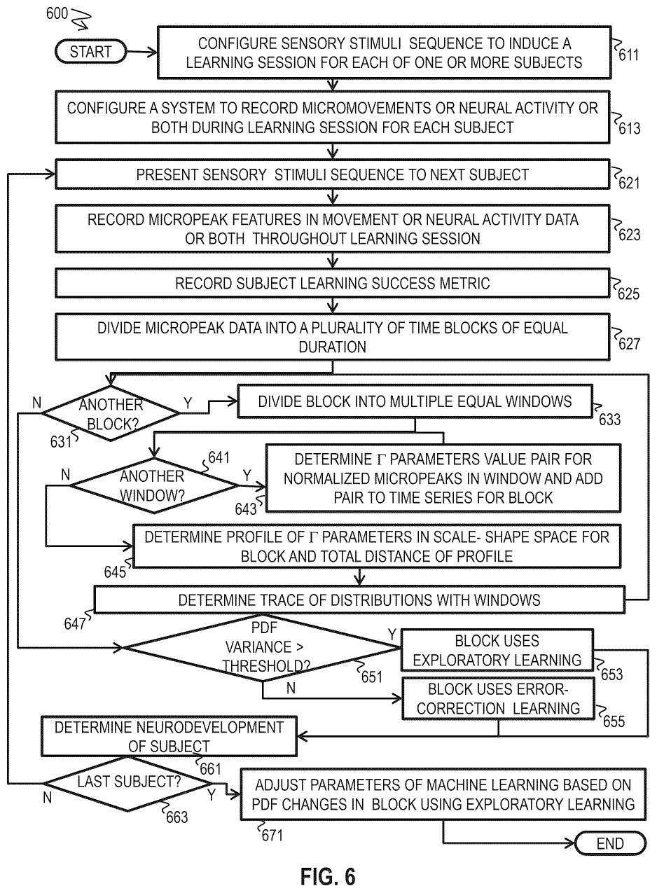

FIG. 6 is a flow chart that illustrates an example of a method 600 to detect and use exploratory learning; according to an embodiment. Although steps are depicted in FIG. 6 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 611, a sequence of sensory stimuli (images, sounds, smells, tastes, contact pressures, or some combination) with repeating components and noise is configured to induce a learning session in a subject. Thus, the sensory stimuli includes visual-motor stimuli in some embodiments. The repeating components can be repeated at random, or with a discernible pattern, or some combination in various embodiments. Thus, the sensory stimuli includes predictable and unpredictable changes. The sensory stimuli is configured to be presented during a learning session to each of one or more subjects. The subjects may be human or animal or autonomous agents, such as robots. Step 611 includes configuring stimulus modules 143, and stimulus subsystem 130, and at least some of learning detection module 150 of system 101 in FIG. 1. In various embodiments, the sequence of sensory stimuli for the learning session is designed to address a particular machine application, such as vehicles in an image of a landscape or skyscape or seascape, or a particular human application such as learning a dance move, or facial expressions to convey emotions, causal relationship between an action consequence and its most probable antecedent, relationship between a movement and its consequence, a most likely next visual/auditory/tactile stimulus in a sequence of stimuli randomly presented, estimating the least likely (most surprising) immediate event in a stream of events, and autonomously figuring out the end goal in a situation, among others.

In step 613, a system, such as system 101 of FIG. 1, is configured to present the sequence of sensory stimuli and to record sub-second movements or neural activity or both during a learning session for each of one or more subjects. Sub-second movements can be detected using video cameras and pose software, as described in previous applications, or accelerometers or position and orientation sensors attached to the subject at one or more locations, including any commercially available sensors and sensors built into ubiquitous mobile devices, such as mobile communication terminals like cellphones, depicted in FIG. 16. Neural activity can be detected using electrodes attached to a subject at various locations including one or more locations used for electroencephalograms (EEGs), auditory brain stem response (ABR) signals, and electrocardiograms (ECGs) among others. In some embodiments, neuromotor activity is measured using medical imaging such as ultrasound used in echocardiograms (EKGs). Or optical sensors such as pulse oximeters Step 613 includes configuring sensor modules 142, video camera 110, and other neuromotor sensors subsystem 120, and at least some of learning detection module 150 in system 101 of FIG. 1, including exploratory learning detection module 155.

In step 621 the sequence of sensory stimuli for the learning session is presented to the next subject of the one or more subjects through stimulus subsystem 130 as driven by stimulus modules 142 configured during step 611. Thus step 621 includes presenting, to a subject, sensory stimuli beginning at a start time and ending at an end time. A stimuli time difference between the start time and the end time is more than 10 minutes and less than 12 hours, and is divided into a plurality of time blocks of equal duration.

In step 623 movements or neural activity are measured by video camera 110 and other neuromotor sensors subsystem 130 and sensor modules 142 during the learning session. These measurements time series are converted to time series of micropeak features (e.g., amplitude, normalized amplitude, prominence, normalized prominence, base width, width at half height, and interpeak time interval). In various embodiments, the sensor data is collected at a rate greater than 100 Hertz (Hz), or greater than 150 Hz, or greater than 200 Hz, or greater than 250 Hertz, or greater than 300 Hz in order to resolve micropeaks.

In step 625, a metric of subject success in identifying the repeating component in each stimulus is measured and recorded. For example, the ability of the subject to identify the correct one of the two or more different components in the current sensory stimulus is measured as a success (e.g., value 1) or failure (value 0), and adding the correct scores. Another example metric is a time to identify the correct component, with the best score being the shortest time. The metric recorded in step 625 is used in a later step to determine the efficacy of the learning approaches taken by the subject during the learning session.

In step 627 the micropeak data (e.g., the time series of micropeak amplitudes, etc.) is divided into one or more blocks of equal duration so that global differences among the beginning, middle and end of the learning session can be deduced. Thus, the stimuli time difference between the start time and the end time is divided into a plurality of time blocks of equal duration. In step 631 it is determined whether there is a block still to be analyzed. If not, control passes to step 651 described below to use all the blocks to determine and use any global differences. If there is a block still to be analyzed, such as the first block, control passes to step 633.

In step 633, the current block is divided into multiple windows of equal duration (window duration in a range from one second to tens of seconds). For example, the block is divided into non-overlapping contiguous windows. In some embodiments, there are gaps between windows, and in some embodiments the windows overlap by less than 100%. In one example embodiment described in section 2, five-second windows overlap in time by 50%. Thus in various embodiments, each window overlaps in time any adjacent window, or each window overlaps any adjacent window in time by 50% of the window duration. Each window has a duration such that an average number of micropeaks in the windows in the block is at least 100 micropeaks. Experience has shown this can be met often by a window duration selected in a range from one second to 10 seconds. Thus a plurality windows of equal duration is formed in each time block of the plurality of time blocks. To characterize variability of windows in a block it is advantageous for each block sto encompass at least 10 non-overlapping windows, and more advantageous to encompass more than 20 windows. Thus, in some embodiments, each block includes at least 20 windows.

In step 641 it is determined whether there is a window still to be analyzed. If not, control passes to step 645 described below to use all the windows' statistics to characterize the current block. If there is a window of the block still to be analyzed, such as the first window, control passes to step 643.

In step 643, the values aw,bw of the Γ parameters shape and scale, respectively, are fit to the empirical histograms of each of one or more of the micropeak features in the current window w, as depicted for example in FIG. 2D. Thus, Gamma function (Γ) scale and shape parameter value pair are determined for micropeak statistics within sensor data observing said subject during each window. The shape-scale values pair for the current window is stored in a data structure, such as exploratory learning statistics data structure 156. The values of aw,bw are used to determine one or more moments of the distribution of that micropeak feature, such as the mean μ, variance σ, skewness, or kurtosis. The values of one or more of those parameters or moments are added to a time series of that parameter or moment for the block to form a trace of that parameter for the block. Control passes back to step 641 to see if there is another window to analyze in the block.

If it is determined in step 641 that there is no other window in the block, then control passes to step 645. In step 645 the time series of the Γ parameters shape and scale is determined for windows w=1 to w=W, where W is the total number of windows in the current block. Thus a profile of Γ scale and shape parameter value pairs are determined during each block. Control passes to step 647. In step 647, the distance between the empirical distributions in successive windows is determined and added to a trace of such distances, as depicted for example in FIG. 3B for the EMD of windows in one block and in FIG. 3C for another block. Thus, a total distance along the profile for each block is determined. Thus, in some embodiments, distance is Earth Mover's distance (EMD) measured using an Earth Mover's metric for distance between distributions characterized by the Γ shape and scale parameter values. Step 647 includes determining the variance of the distances in the trace for each block. In some embodiments, instead of the pdf distance between windows in each block, the change in the shape or scale parameter values, or one or more moments derived therefrom (using Equations 3a through Equation 3d), are traced window to window in each block, and the variance is the variance in the shape or scale parameter values or one or more moment values of changes thereof between successive windows. Thus, a distance along the profile between each pair of successive windows within each block is determined. Control then passes back to step 631 to process the next block.

If it is determined in step 641 that there is no other block in the learning session, then control passes to step 651. In step 651, it is determined whether the variance of the trace (pdf distances or shape or scale parameter values or moments derived therefrom or changes therein) is greater than some threshold variance that defines exploratory learning. Thus, it is determined that the subject is engaged in exploratory learning during each block in which variance in that block exceeds a threshold. In some embodiments, the threshold is about 2.6 which divides the two types of learning depicted in FIG. 5. In some embodiments, the threshold is a percentage of the total range of scale between the extreme points in the log-log shape-scale plots, e.g., a percentage of the total distance represented by the double arrow in FIG. 2G. In various embodiments, that percentage is 75% or 50% (median) or 25%. For example, when the threshold is 50% of that maximum pdf distance observed in the block, then the subject is primarily in the LUQ, which is a characteristic of the exploratory learning observed in experiments. It has been found that this divide between exploratory learning and error correction learning predicts a divide between those remembering the regularity and scoring high on the memory metric (the error-correction type B learners) and those without a recall of the regularity who score low on the memory metric (the exploratory type A learners).

In some embodiments, step 651 includes comparing learning success metrics in a block to the pdf distance (e.g., EMD) or shape or scale or one or more moments mean or variance among windows in the block. Periods of low learning success metric are indicative of exploratory learning, so the associated pdf distance or shape or scale or moment mean or variance forms a range of means or variances that characterize exploratory learning. By the same token, a range of means or variances associated with high learning success are associated with later stage error-correction learning. A threshold is then selected that separates the two ranges, such as a mean or variance halfway between the centroids of the means or variances of the two groups (blocks with high success metrics and blocks with low success metrics). It is noted that using exploratory learning with low success can be an effective strategy in discovering patterns, much as a gradient descent optimization is occasionally displaced randomly to avoid descending into a sub-optimal local minimum error. In some embodiments, the threshold is a percentile of Γ scale values along the profile, such as the 50^{th} percentile, so that the variance in the block resides in one quadrant.

In other embodiments, the threshold is determined by selecting a median value of the variance determined in all the blocks among all the subjects in a learning session with mixed random and discernable patterns in the sequence of sensory stimuli. In other embodiments, the threshold is determined by using cluster analysis to see if the variance forms more than one cluster among one or more or all the blocks of one or more or all the subjects tested in a learning session with mixed random and discernable patterns in the sequence of sensory stimuli. The threshold would then be halfway between the midPoint of those two clusters. In embodiments like in this paragraph, in which the threshold is not defined until several subjects are measured during the learning session, steps 651, 653 and 655 are moved after the last subject is determined in step 663.

If it is determined in step 651 that the variance is greater than the threshold, then in step 653 it is determined that the block uses exploratory learning. If it is determined in step 651 that the variance is not greater than the threshold, then in step 655 it is determined that the block uses error-correction learning. In either case, control passes to step 661.

In step 661, a neurodevelopment condition of the subject is determined based on the use of exploratory learning or not. For example, if a subject uses exploratory learning in too many blocks, then that subject can be considered not as advanced as a subject of the same age who quickly converges on error-correction learning. Thus, a neurological condition of the subject is determined based upon a percentage of time during the presentation of the sensory stimuli during which the subject is engaged in exploratory learning. In embodiments in which neurodevelopment differences are not defined until several subjects are measured during the learning session, steps 661 is moved after the last subject is determined in step 663.

In step 633, it is determined whether the last subject has been exposed to the learning session. If not, control passes back to step 621 to present the sequence of sensory stimuli to the next subject and the steps following 621. If so, control passes forward to step 671 or ends.

In step 671, the parameters of a machine learning algorithm being taught on training data are adjusted from one early training set iteration to another based on the change in pdfs observed during blocks categorized as exploratory learning. It is expected that such exploratory learning will be jumping frequently relatively large distances across quadrants between exponential-like distribution and Gaussian distributions. Thus adjusting parameter values for a machine learning algorithm is performed using exploratory learning, wherein exploratory learning comprises a sequence of probability density functions used by a subject engaged in a block of exploratory learning.

As a result of the method 600 described here, one finds neural correlates for two types of learners in a visuomotor task: narrow variance learners, who retain explicit knowledge of the regularity embedded in the stimuli and use closely related pdfs. They seem to use an error-correction strategy steadily present in both stable and unstable environments. This strategy can be captured by current optimization-based computational frameworks. In contrast, broad variance learners emerge only in the unstable environment. Local analyses of the moment-by-moment fluctuations, naïve to the overall outcome, reveal an initial period of memoryless learning, well characterized by a continuous gamma process starting out exponentially distributed whereby all future events are equally probable, with high signal (mean to variance ratio MVR).

The empirically derived continuous Gamma function Γ parameters smoothly converge from memoryless exponential to predictive Gaussian distributions comparable to those observed for the error-corrective mode that is captured by current optimization-driven computational models. We coin this initially seemingly purposeless stage “exploratory learning.” Globally, we examine a posteriori the changes in the empirically estimated stochastic distributions. We then confirm that the exploratory mode of certain learners, free of expectation, random and memoryless, but with high signal, precedes the acquisition of the error-correction type which exhibits smooth transition from exponential to symmetric Gaussian distributions. This early naïve phase of the exploratory learning process has been overlooked by current machine learning models driven by expected, predictive information and error-based learning. This work demonstrates that (statistical) learning is a highly dynamic and stochastic process, unfolding at different time scales, and evolving distinct learning strategies on demand.

### 2. EXAMPLE EMBODIMENTS

Various examples of the detection of exploratory learning are described herein. In these example embodiments, the term micromovement spikes (MMS) is used to describe both sub-second movement data retrieved from movement sensors, including video, and sub-second variations in neural activity often measured electronically, e.g., as electroencephalograms (EEGs). Thus, within this section MMS corresponds to micropeaks.

In any case, all such data can be reduced to micropeaks and normalized micropeaks and the statistics of features of such normalized or un-normalized micropeaks can be processed as described above to detect and use exploratory learning. The material in this section has been published after the priority date of this application. In order to better map the publication to this application some of the terms are adjusted herein to more closely map to the example method of FIG. 6, except that “MMS” and “micropeaks” will be used interchangeably. Similarly, the terms “participants” and “subjects” are used interchangeably. In addition, the terms “stochastic signatures” and “pdf distributions” are used interchangeably.

In various experiments, fluctuations of a continuous neuromotor signal measured in a subject, recorded during a learning task (such as a visual search), are analyzed. During precise time stamping of the events in the data acquisition system and the use of stable and unstable implicit-learning environments, one empirically estimates anew, short-time window by short-time-window (e.g., moment by moment), the probability distribution function (pdf) that best fits micropeaks in the data. Over time one obtains a continuous family of pdfs describing the overall learning process. This process allows these micropeak fluctuations, which are often discarded as gross data, reveal the primordial way of curious, exploratory learning. Exploratory learning precedes self-discovery of regularities that become a goal, wherein deviations from that goal eventually define the error in an error-correction type of learning. This reframes statistical learning from the point of view of a developing, nascent motor system that spontaneously transitions from purposeless to purposeful behavior.

In the following descriptions in this section, exploratory learning is variously characterized as naïve, open to surprise, memoryless, non-predictive (all future events equally probable), signal to variance (noise) is high (in pdf distance between windows), exponential distribution in brain activity (smaller shape a), suitable to unstable environments, broad variance (in pdf characteristics among different windows), primarily utilized in unstable environments.

In contrast, in the following descriptions in this section, error-corrective learning is variously characterized as goal-oriented, having precepts, using memory, akin to current supervised and unsupervised machine learning, having predictive codes, Gaussian distribution in brain activity (larger shape parameter a), narrow variance, finds regularity embedded in both stable and unstable stimuli environments.

In the following descriptions in this section, the transition from exploratory to error-corrective learning is variously characterized as smooth, autonomous self-discovery, learn on own (unsupervised), appropriate for use in initial stages of machine learning (e.g., initialization of machine model parameter values), on global time scale of entire learning session.

### 2.1 EEG During Visual Search Task Learning

This experiment involving human participants was reviewed and approved by the Institutional Review Board of Tel Aviv University. The participants provided their written informed consent to participate in this study. Behavioral and Event Related Potentials (ERPs are the EEG waveforms averaged over many trials) analyses of these data were previously published. Here we focus on the continuous EEG signal, without taking data epochs and averaging data parameters under theoretical assumptions of normality, linearity, and stationarity. Instead, we empirically estimate the continuous family of PDFs that in a maximum likelihood (MLE) sense, best fits micropeaks that are traditionally discarded as superfluous gross data. This novel approach enabled us to isolate phenomena that cannot be observed when data is analyzed with conventional methods, leading to the uncovering of entirely new results.

Participants. Data from 70 participants (48 female, mean age, 23.7) was analyzed in this study comprising three groups: 24 in a random repeat learning session; 23 in a correlated repeat learning session; and 23 in a mixed random and correlated repeat learning session. There were no differences in age or gender between the three experimental groups. All participants gave informed consent following the procedures of a protocol approved by the Ethics Committee at the Tel Aviv University. Two participants (one in the mixed group and one in the correlated group) were removed from the analyses due to incomplete data, e.g., their EEG recording started late, missing the first few trials. As we focus here on continuous data analyses of the full learning experience, these two subjects were excluded.

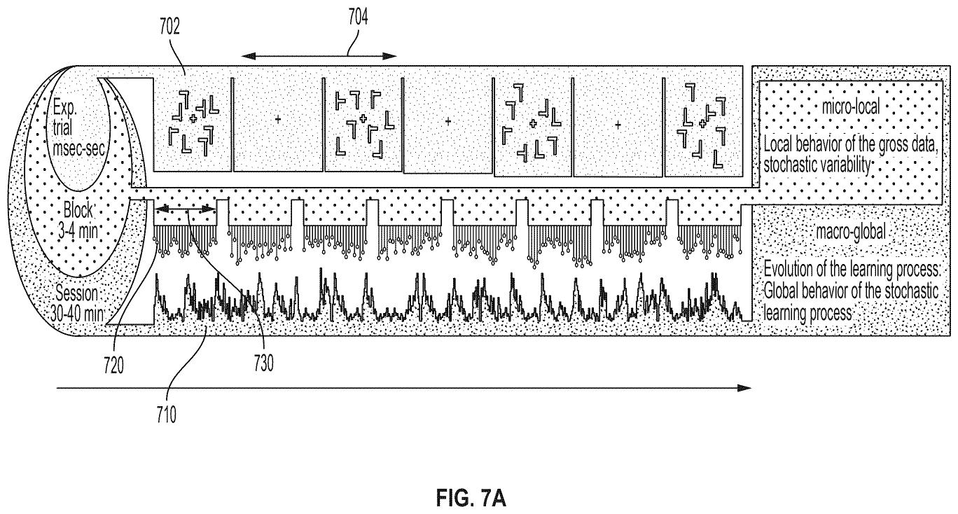

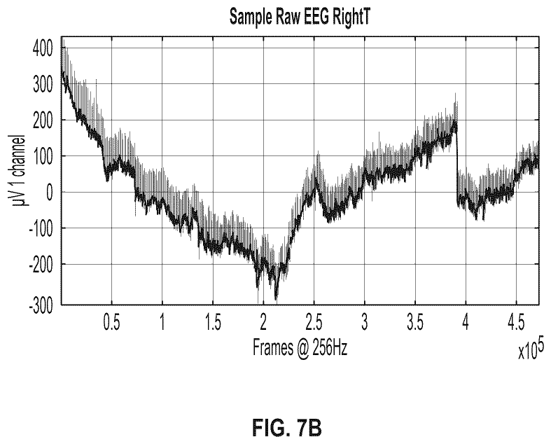

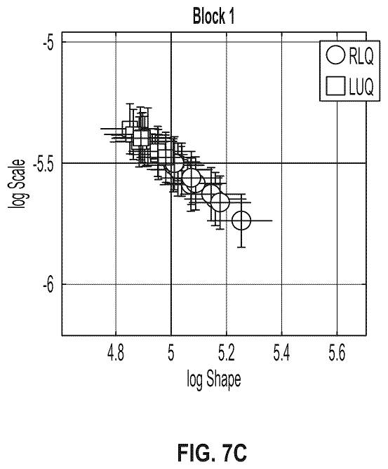

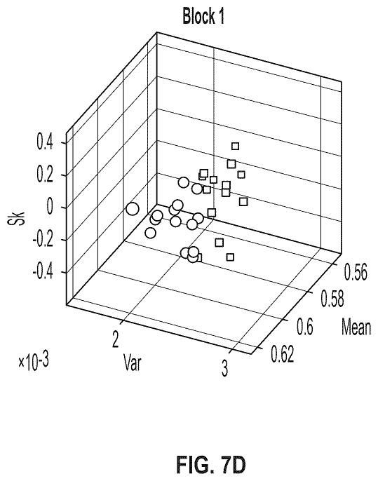

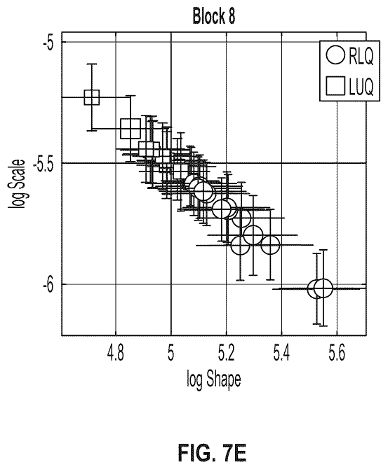

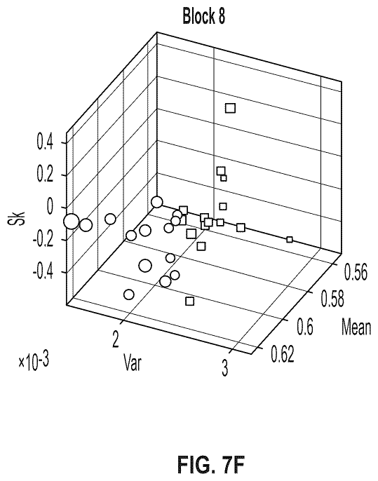

Stimuli and Procedure. FIG. 7A is a block diagram that illustrates a schematic relationship among processes in a learning session, according to an experimental embodiment. FIG. 7B through FIG. 7F are plots that illustrate examples of micropeak distributions during experimental learning sessions by one or more subjects, according to an embodiment. The EEG signal from each subject was recorded during a visual search task. This task was followed by an explicit memory test during which EEG was not recorded. A more detailed account of the procedure can be found in Vaskevich et al. (2021). The sequence of sensory stimuli in a learning session is represented in FIG. 7A which is a block diagram that illustrates example visual search tasks in a sequence progressing from bottom to top in time in the direction of the arrow in FIG. 7A. The total duration of a learning session lasted tens of minutes, e.g., 30 minutes to 40 minutes.

Upon presentation of a visual image stimulus 702 depicted along the top above the time line arrow, each subject is to respond by indicating detection of a left T or a right T. The presentation of the image and the response by the subject constitutes one trial 704 and typically lasted a few milliseconds up to several seconds. The resulting EEG recordings were converted to normalized micropeaks 710 as indicated along the bottom of the figure closest to the timeline arrow. The statistics of the micropeaks were computed within 5 second 50% overlapping windows 120, each window represented by a line at the window start time with a height indicative of some measure of the stochastic signature in the window, in arbitrary units, for purposes of illustration only. The windows were collected into a block 730 of 3-minute to 4-minute duration and represented by grey rectangles in FIG. 7A.

The repeatability of the left and right Ts was either random for group 1, or correlated in a pattern of some discernable type for group 2, or a mixture of the two types of repeats for group 3. Memory of the discernible repeat pattern was used as a learning success metric.

Visual stimuli in the visual search task (and in the explicit memory test described below) were white T's as the target and Ls as distractions. All stimuli were made up of two line segments of equal length (forming either an L or a T). From a viewing distance of approximately 60 centimeters (cm), each item in the display subtended 1.5°×1.5° of visual angle. All items appear within an imaginary rectangle (20°×15° on a grey background with a white fixation cross in the middle of the screen (0.4°×0.4°. Target Ts appeared with equal probability on the right or left side of the screen.

Participants searched for a rotated T (target) among heterogeneously rotated Ls (distractors) while keeping their eyes on the fixation cross. Each trial began with the presentation of a fixation cross for 2100, 2200, or 2300 milliseconds (ms), randomly jittered, followed by an array of one of two possible targets (left or right rotated T) among seven distractor Ls. Participants were instructed to press a response key corresponding to the appropriate target (left rotated T or right rotated T) as fast as possible.

The implicit learning goal of the task was to be accurate as fast as possible. Predicting any repeat patterns assists this implicit goal. Learning the repeat patterns, if any, was the self-discovery to be learned. Each participant was randomly assigned to one of the three groups, with the degree of regularity in the task varying along a gradient. At one extreme the participants searched for the target within a highly predictable environment where predefined spatial configurations of target and distractors (layouts) were repeated from trial to trial (correlated group). Presumably, the embedded regularity can be easily and systematically confirmed by the subject. At the other extreme, participants experienced the least amount of regularity, as from trial to trial, the layouts of the display were generated randomly (random Group). For the third group, consistent and random layouts were mixed throughout the task (mixed group). Any regularity is cumulatively built from random guesses and confirmations, thus creating the ground for self-emergence of the overall goal or purpose of the task. This task is ideal to investigate the dynamic progression of statistical learning.

The gradient of predictability enables one to examine, moment by moment, each moment captured in a short duration window, stochastic variations in learning between environments that differ in the reliability of predicting and confirming a guessed regularity. Depending on the group, the visual search contained the consistent mapping condition (correlated group) the random mapping condition (random group), or both (mixed group). In summary, the three groups corresponded to predictable predictability (consistent group), predictable unpredictability (random group) and unpredictable predictability (mixed group). Of particular interest was learning in the mixed group relative to the other two groups. For the predictable mapping condition, spatial configurations of targets and

distractors were randomly generated for each participant (8 layouts for the mixed group and 16 layouts for the correlated group). In the random mapping condition, targets and distractors appeared in random locations throughout the task. The order of layouts was randomized every 16 trials (in the case of the mixed group 16 trials correspond to 8 consistent and 8 random trials presented in a random order). The identity of the target (left or right rotation) was chosen randomly on each trial and did not correlate with the spatial regularity.

Participants each completed 512 trials in the experiment. Only correct trials were included in the analysis.

Explicit memory test. Participants were not informed of the regularity in the visual search task. Upon completing the task, participants in the mixed and correlated groups (when the task contained regularity) completed an explicit memory test, designed to reveal explicit knowledge of the regularity: participants saw the layouts that were presented to them during the search task mixed with new, randomly generated layouts. For each layout participants had to indicate whether they have seen the layout during the visual search task or not. An Explicit Memory Test (ET) score (hit rate/false alarm rate) that is considered to reflect each participant's explicit knowledge of the regularity was computed, so that higher scores correspond to better explicit knowledge.

EEG recording. EEG was recorded inside a shielded Faraday cage, with a Biosemi Active Two system (Biosemi B.V., The Netherlands), from 32 scalp electrodes at a subset of locations from the extended 10-20 system. The single-ended voltage was recorded between each electrode site and a common mode sense electrode (CMS/DRL). Data was digitized at 256 Hz. Continuous recordings were used, without averaging epochs of the data. In this experiment, we use only the electrodes that reflect unconscious activity and do not reflect strong eye muscle activity either through blinking or the jaw movement which are highly controlled by the participants in this kind of task. The analyzed subset of electrodes, identified using the conventional EEG naming convention, were Fp1, Fp2, AF3, AF4, F3, F4, F7, F8, Fz, FCz, C3, C4, Cz, T7, T8, P1, P2, P3, P4, P5, P6, P7, P8, Pz, PO3, PO4, PO7, PO8, POz, O1, O2, and Oz. These include all the electrodes that were previously analyzed (P7, P8, PO3, PO4, PO7, PO8, C3, C4). The EEGLAB PREP pipeline was used to clean the EEG signals. One signal was selected to represent the subconscious brain activity associated with learning. That one electrode was based on the hub electrode in a network representation.

Cross-Coherence Analyses and Network Representation. The statistical analyses described in the next sections were done for a hub channel, chosen continuously for each time window (5 seconds of recording) with 50% overlap of the sliding window. Here is described the process by which these hub channels were selected. Based on previous work with the same approach the data were bandPass filtered to pass the band from 13-100 Hz using IIR filter at 20th order. Two sample leads, taken pairwise across all sensors of the EEG cap were then used to instantiate the analyses. Cross-coherence was used to quantify the similarity between any two leads. For each pair, the maximal cross-coherence was obtained, with corresponding phase and frequency values at which the maximum was attained. The maximal cross-coherence matrix was used as an adjacency matrix to build a weighted undirected graph representation of a network. Next, network connectivity analyses were used to obtain the maximum clustering coefficient representing the hub within each time window at the selected frequency band.

FIG. 7B is a plot that illustrates an example of the EEG signal from one hub channel determined through network connectivity analyses. The horizontal axis indicates time in 10⁵ frames sampled at 256 Hz; thus, the horizontal axis represents a data duration of about 4.7×10⁵ frames, or about 1800 seconds equal to 30 minutes. The vertical axis indicates EEG signal amplitude in microvolts (μV).

The stochastic signatures of the moment-by-moment fluctuations in neural activity (i.e., the window by window pdf distributions) were then tracked in each overlapping window for the identified hub. To obtain the MMS of the EEG-hub biorhythmic signal for each participant, the micropeaks of the original EEG-hub waveform were taken, the empirical distribution of the micropeaks were derived, and using the empirically estimated mean, the absolute deviation of each time point in the EEG-hub time series from the empirically estimated mean were obtained.

The empirically estimated mean voltage amplitude was used in these computations to track the moment-by-moment fluctuations away from this empirically estimated mean. This builds a time series of micropeaks (e.g., micromovement spikes, MMS) which consists of periods of activity away from the mean interspersed with quiet periods of near-mean activity. Importantly, the original times where those fluctuation peaks occurred were retained. The normalized MMS trains were built using the deviations from the mean amplitude using Equation 2.

Sweeping through the normalized MMS trains, the values of the peaks (ranging now between 0 and 1) are gathered into frequency histograms for windows of 5 seconds with 50% overlap between each two consecutive windows. We explored between 1- and 5-second-long windows (with 50% overlap) and settled on 5 seconds as the minimal time unit that gave us acceptable 95% confidence intervals in the empirical estimation of Γ shape and scale values, requiring 100 peaks or more.

Local Analyses: Empirical Estimation of T′ Scale and Shape parameters. The histograms of normalized micropeaks in each window were fit by values of the Γ parameters shape and scale using Maximum Likelihood Estimation (MLE). This gives us a local estimation (at each window) of the stochastic process, i.e., the stochastic signature. An example of frequency histogram fit was shown in FIG. 2D. The Γ shape and scale parameter values aw and bw, respectively, of each window w, were plotted for all windows in the first block of an experiment on the Γ parameter log-log plane in FIG. 7C. The 95% confidence intervals are indicated for each point. The horizontal axis indicates the log of values for the shape parameter and the vertical axis indicates the log of values for the scale parameter. The median point and the resulting right lower quadrant (RLQ) and left upper quadrant (LUQ) are also depicted, with points in the former plotted as open circles and points in the latter plotted as open squares. FIG. 7D is a 3D plot that illustrates an example of the mean, variance and skewness moments associated with each of these points in shape and scale space, using open circles to represent the points in the RLQ and open squares representing points in the LUQ. FIG. 7E and FIG. 7F correspond to FIG. 7C and FIG. 7D, respectively, but for the 8th block of the experiment for this subject.

These plots confirm previous work, as the log values of the Γ parameters have been found to be useful for representing MMS derived from human biorhythmic data registered from the face, eyes, whole body, heart, EEG, ABR and fMRI signals.

FIG. 7C shows the log-log Γ parameter plane with a division into quadrants that reflect different empirical properties of the stochastic process. We take the median of the shape values and the median of the scale values and draw a line across each axis to break the Γ parameter plane into quadrants that shift from window to window. Quadrants reflect the evolution of the stochastic process and the quadrant boundaries, defined by median shape and median scale values for the window, define a local threshold that indicates substantial changes as empirical pdfs swing from one quadrant to the other. FIG. 7D shows the corresponding moments space following the symbol code of FIG. 7C whereby points that fall on the right lower quadrant (RLQ) are those representing symmetric distributions with low VMR (low dispersion), while those in the left upper quadrant (LUQ) represent distributions closer to the exponential range and having high VMR.

Here is explained the empirical meaning of the Γ parameter plane. In one block, one is viewing the local (short term) variations in pdfs of a few minutes. Generally, at the leftmost extreme, when the Γ shape value is 1, one has the special case of the memoryless exponential distribution (no empirical shape-scale points appear in this range for this example). This is the case of having a random process whereby events in the past do not inform more about events in the future than current events would. All future events are equally probable. The information is coming from the here and now. Such distributions are typical for the motor patterns at the start of neurodevelopment. Around 4-5 years of age, when the subject is (on average) mature enough to start schooling, receive instructions, and sustain longer attention spans, a transition into heavy tailed distributions is observed. By college age these distributions are tending to Gaussian, so that the shape parameter is at the other extreme of the shape axis on the Γ parameter plane and the MVR is at its highest value. Prior work has also revealed that in systems where maturation is compromised (e.g., autism across the lifespan) these stochastic signatures remain in the exponential range, randomly relying on the here and now and manifesting very low MVR. In this case, the system does not progress into acquiring a predictive Gaussian distribution.

Global A Posteriori Stochastic Analyses of Distribution Shapes. Relying on the linear relationship between log shape and log scale in each block, one of the parameters, e.g., shape, can be used to track the global changes over all blocks in the experiment. Using the pdf distributions of micropeaks in an entire block, rahter than mutiple windows, i.e., when the window size equals the block size, one can detect the full stochastic profile, a posteriori, at a global time scale, e.g., block by block across the entire session.

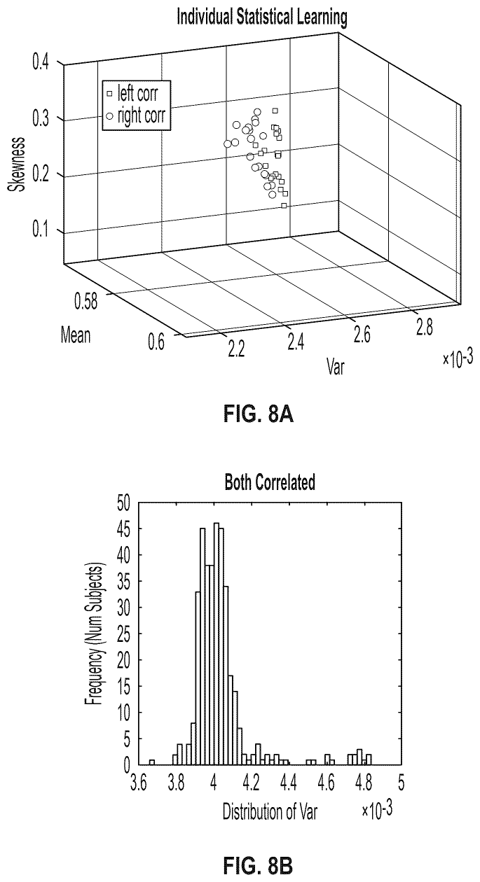

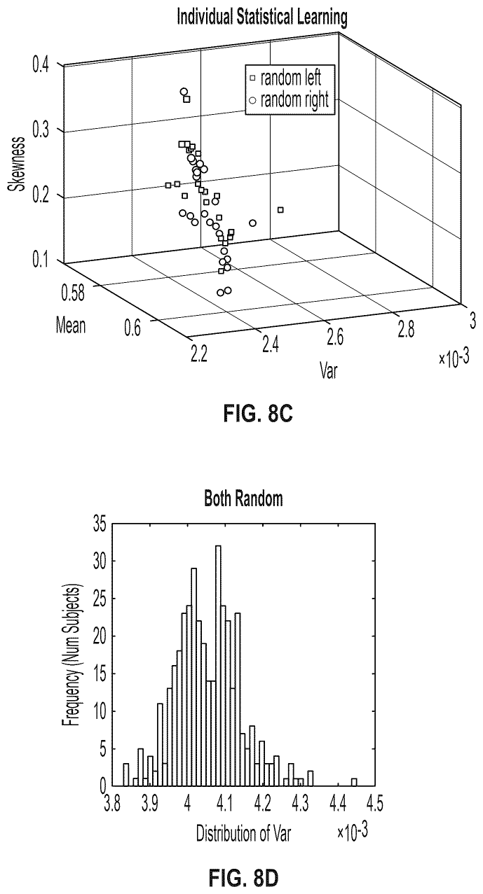

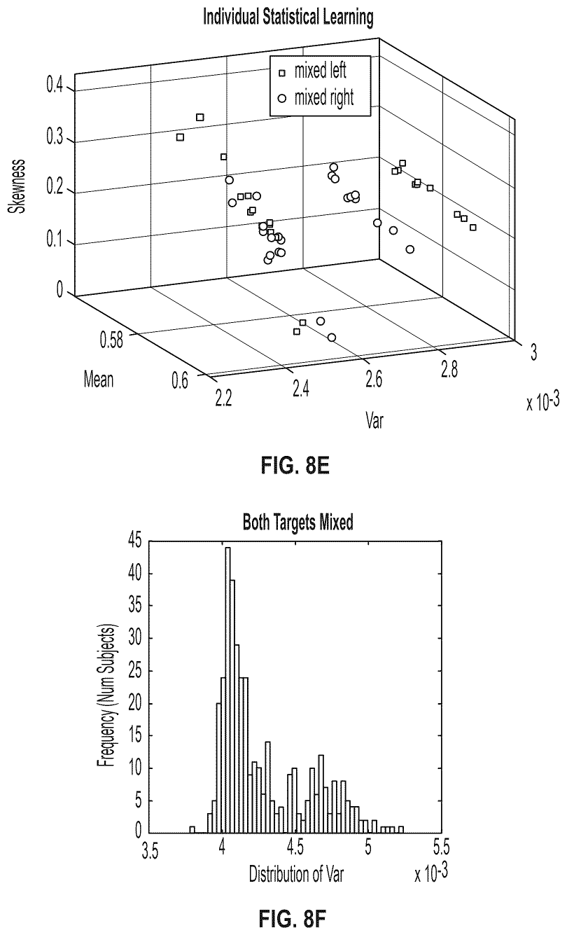

FIG. 8A through FIG. 8F are plots that illustrate examples of differences between micropeak distributions during different experimental learning sessions by one or more subjects, according to an embodiment. Empirically estimated moments using Equation 3a through Equation 3d for a Γ fit to the distribution micropeaks span a parameter space whereby each participant represents a point. The coordinates are the mean (x-axis), the variance (y-axis), the skewness (z-axis) of the micropeak distributions across the whole block. The symbol represents the target orientation (open square for left and open circle for right right) of the correctly identified target.

FIG. 8A shows the moments for the correlated group, for which layouts are consistent from trial to trial, presenting a stable learning environment. FIG. 8B shows the corresponding frequency histogram of the micropeaks across trials, target types and participants. FIG. 8C shows the moments for the random group, for which layouts are generated randomly from trial to trial, presenting a stable learning environment where no regularity is present. FIG. 8D shows the corresponding frequency histogram of the distribution of the micropeaks across trials, target types and participants. The distribution of micropeak frequency is much wider than in FIG. 8B.

FIG. 8E shows the moments for the mixed group whereby trials intermix random and correlated conditions, presenting a relatively unstable learning environment. In this group two distinct subgroups of participants emerge. FIG. 8F shows the corresponding frequency histogram of the micropeaks across trials, target types and participants. The distribution of frequency appears bimodal, representing the two distinct sub-groups.

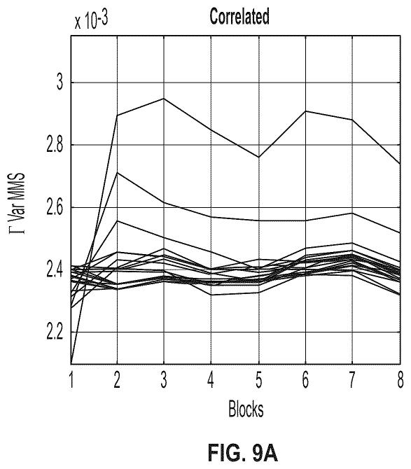

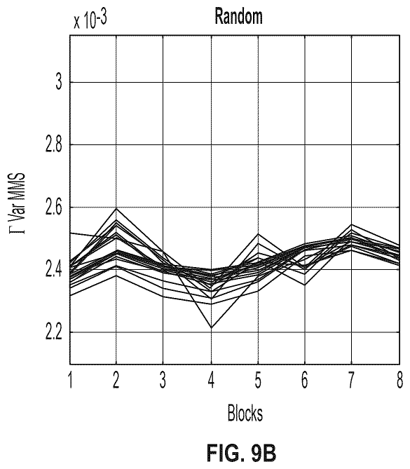

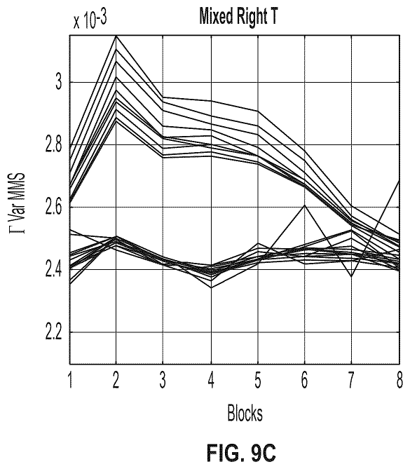

Global Analyses: Evolution of empirical PDFs over a learning session. FIG. 9A through FIG. 9C are plots that illustrate examples of temporal evolutions of variances in micropeak distributions during different experimental learning sessions by one or more subjects, according to an embodiment. The empirically estimated variance is based on a T fit (equal to the scale parameter value b of the fit as shown by Equation 4) of the micropeaks in each block for each subject as measured by scale parameter values or one or more moments derived therefrom . . .

The correlated group for the correct right rotated Ts (FIG. 9A) shows relatively constant levels of variance in the values of the scale parameter for all windows in the block and stable learning throughout the 8 blocks of the experimental session. Each profile represents the trajectory of a participant within the group. In FIG. 9A two participants are outliers. The random group (FIG. 9B) also shows relatively constant levels of variance and stable learning throughout the 8 blocks of the experimental session. Similar patterns occur for the correct left rotated Ts for both correlated and random groups.

For the mixed group (FIG. 9C which is a repeat of FIG. 5 for convenience), two subgroups (also called types herein) are revealed for both right- and left-oriented targets. A first subgroup (type A) with high variance and broader range of scale variance values separate from those in a second subgroup (type B) with lower variance and narrower range of values throughout the experimental session. However, both subgroups converge to similar variance levels towards the 8th block at the end of the learning session. The correct left rotated target for the mixed group shows different absolute high variance values but similar convergence to lower variance at the later blocks.

The two subgroups, broad-variance type A and narrow-variance type B of the mixed group did not differ in reaction times or accuracy during trials, suggesting that all participants were able to reach the same level of online performance. Instead, they were differentiated by their explicit knowledge of the regularity imbedded in the task, as reflected by their memory scores in the explicit memory test. The 10 subjects in the broader variance type A, M=0.94, SD-0.4 vs. 13 subjects in the narrow variance group type B, M=1.52, SD=0.75, p<0.01 nonparametric Wilcoxon ranksum test.

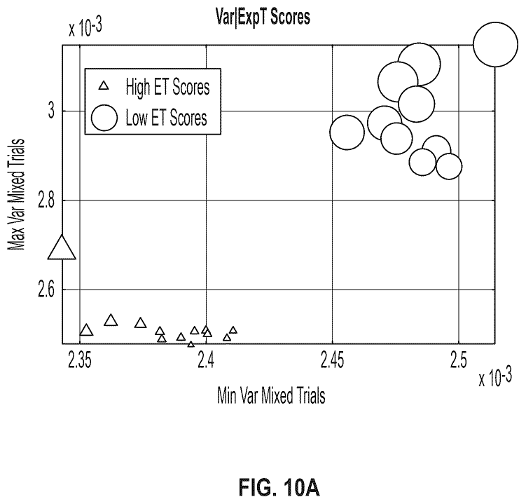

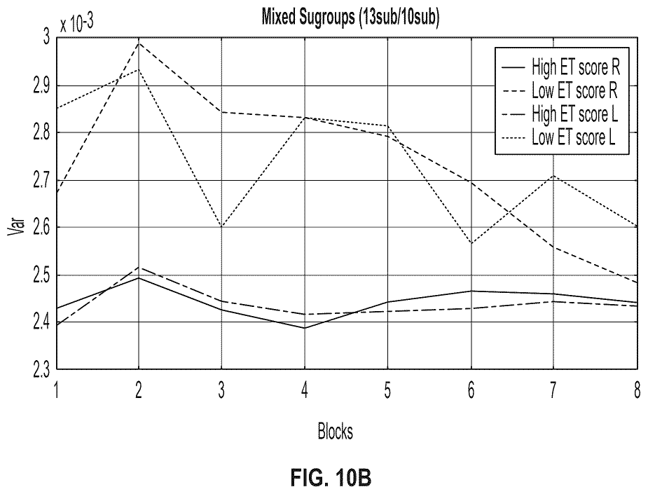

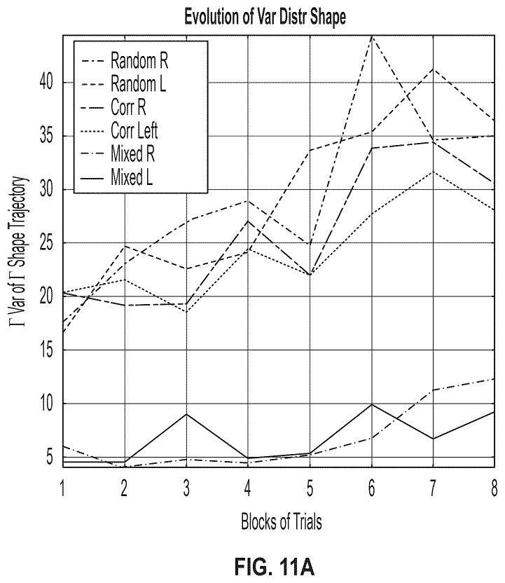

FIG. 10A and FIG. 10B are plots that illustrate learning success metrics of subjects engaged in experimental learning sessions relative to their evolutions of micropeak variances, according to an embodiment. Self-emerging types in the mixed group are differentiated by the scores of the explicit memory test used as learning success metric. In FIG. 10A, the horizontal axis indicates the minimum value of the variance, while the vertical axis indicates the maximum value of the variance for each participant. Thus, the graph depicts the full range of variance values. The size of the marker is proportional to the explicit memory test score and the symbol represents the type, with no overlapping between the two sets of participants. FIG. 10B illustrates examples of empirically estimated Γ scale parameter b (indicating the VMR in the distribution of window distribution values as given by Equation 4) block by block as in FIG. 9C, for the two subgroups of the mixed condition. Unlike in FIG. 9, here the variance of all the participants of one type is averaged. The type with less explicit knowledge (lower scores on the explicit memory test, e.g., ET score M=0.94, SD=0.4) starts out with higher variance (broad-variance type A), eventually converging to the much lower variance level of the subgroup that showed higher explicit knowledge of the regularity (ET score M=1.52, SD=0.75) (narrow-variance type B).

Thus, type A with broader range of variability showed low test scores, thus exhibiting less explicit knowledge of the regularity. In contrast, type B with the narrow, steady variability, gained a higher level of explicit knowledge, as reflected in higher explicit memory test scores. We consider the process showing higher variance with low explicit memory score (type A) “exploratory mode.” In contrast, we consider the process showing lower variance and high explicit memory score “error-correction mode” (type B). Here the mode refers to a type of learning.

For completeness, the memory scores of the correlated group were also examined. Overall, memory scores (M=1.37, SD=0.9) were like the high scores observed in type B of the mixed group. This result is consistent with the similar stochastic learning signatures of the correlated group and this high memory type (observed in the variance trajectories of FIG. 9A and FIG. 9B). We here infer that, because the regularity in the correlated group was highly reliable with layouts repeating on all trials, it seems that all participants quickly reached some minimal level of explicit knowledge, e.g., within one block, therefore no differentiated subgroups emerged.

Global time evolution based on micropeak variations. The next results provide a stochastic characterization of these two fundamentally different types of learning which, nevertheless, converged in block 8 to a similar micropeak variance range.

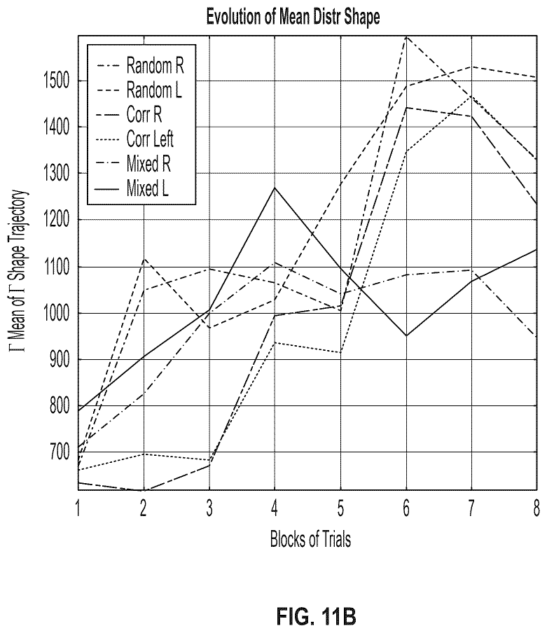

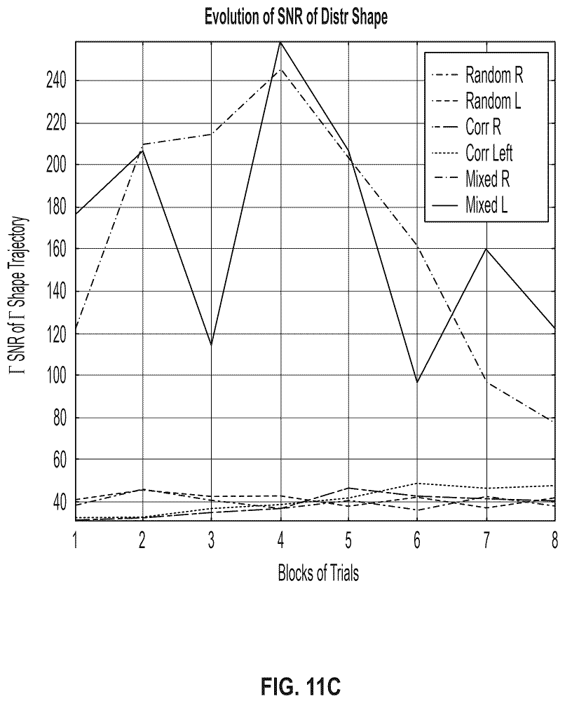

Shape parameter values for micropeaks can also be tracked among all blocks, and their moments can be computed empirically. Among the moments of the distributions of the shape parameter, the variance of the shape parameter aw revealed the separation of the mixed group from the random and from the correlated groups. FIG. 11A through FIG. 11C are plots that illustrate temporal evolution of Gamma function (Γ) statistics of micropeak distributions during different experimental learning sessions by one or more subjects, according to an embodiment. Learning evolution taken globally across participants and full session, shows the unstable environment (mixed group) to provide the most efficient conditions for learning, as indicated by the highest MVR. FIG. 11A is a plot tracking, block by block, the empirically estimated variance of the shape values of the MMS amplitudes in each block, for each type of stimulus and target. Correlated and random groups trend upward with a steeper rate for correlated, while the mixed group stabilizes after ½ the session. The variance separates the correlated and random groups from the mixed group, with a marked reduction on the variability of shape values and an overall trend to increase the variability in shape values towards the final blocks. FIG. 11B is plot tracking, block by block, the empirically estimated mean value of the shape values from the fluctuations in MMS amplitudes. FIG. 11C is plot tracking the signal to noise ratio (mean/variance) then shows the highest signal for the mixed trials, with a downward tendency after ½ the total session.

Thus, a distinction is observed for the mean parameter of the shape values. As such, the MVR shows the highest signal content for the mixed group. For both the correlated and random groups, the mean shape has an increasing trend, consistent in both cases for the right-and left oriented targets. However, in the mixed group, there is an initial increase in the mean shape that decreases and stabilizes by the 4th to 5th block, at much lower values of the variance, so that the MVR of the mixed group is much higher than that of the random or correlated groups. This elevated MVR indicates that the mixed environment is much more effective for learning than environments that contain purely random or purely correlated trials alone. Its information content is higher.

We show the stochastic shifts of each of the error correction (lower Γ shape variance and higher explicit memory test score) and exploratory (higher Γ shape variance and lower explicit memory test score), as they unfold across the blocks. The empirically estimated Γ shape parameters of the subgroup with high explicit 514 memory scores (type B) starts in the symmetric Gaussian range but trends down and converges towards the skewed, heavy tailed distributions.

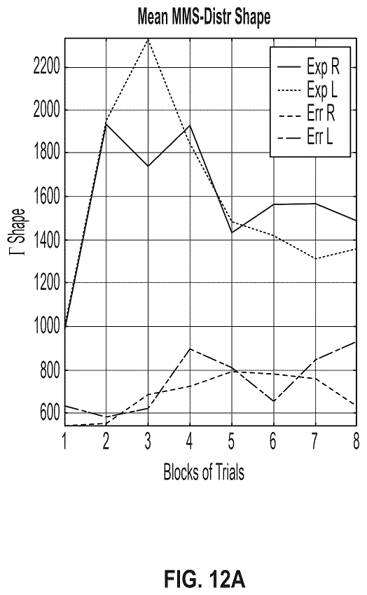

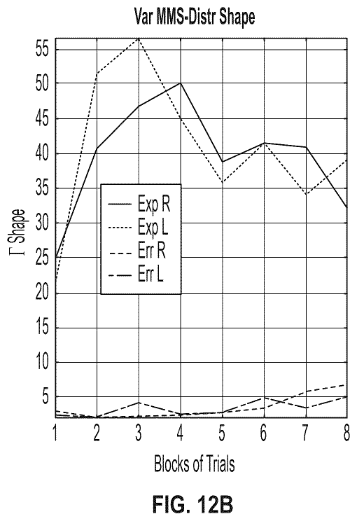

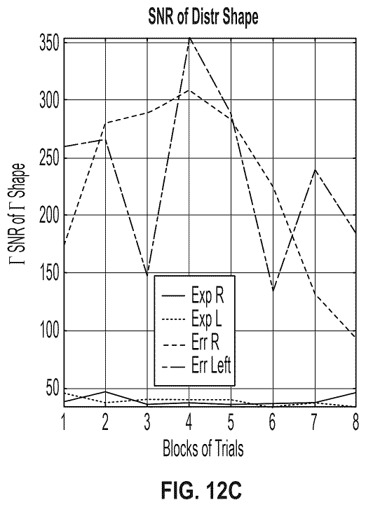

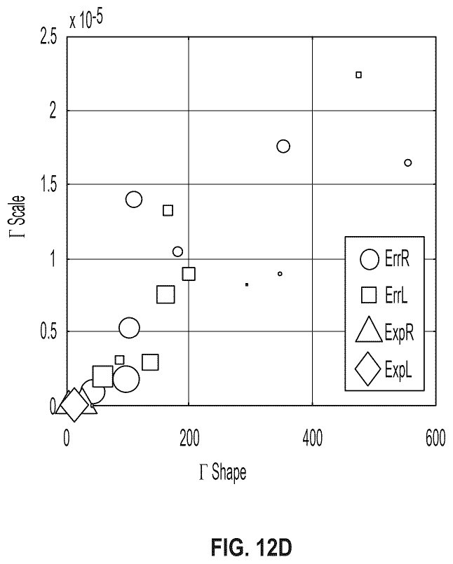

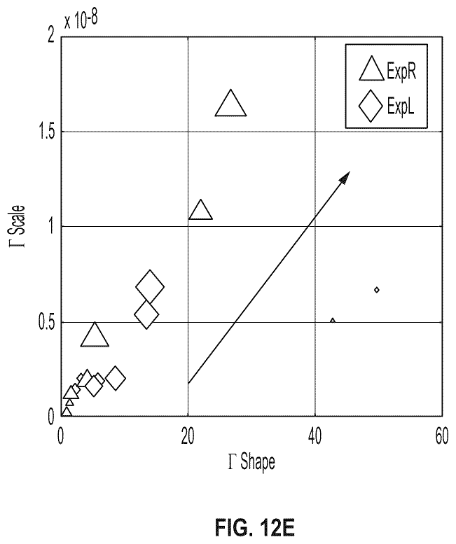

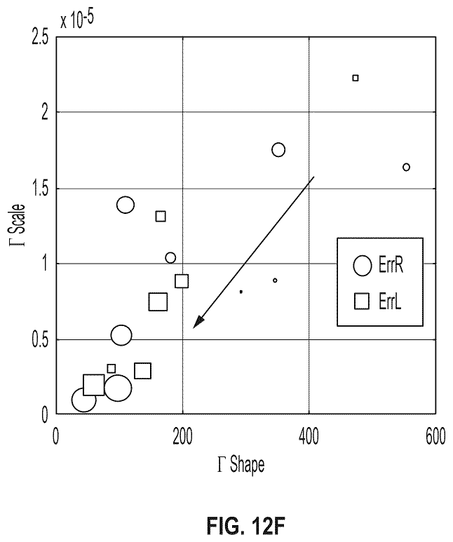

FIG. 12A through FIG. 12F are plots that illustrate temporal evolution of Gamma function (Γ) parameters of micropeak distributions for different shapes and different learning types during experimental learning sessions by one or more subjects, according to an embodiment. Stochastic characterization of exploratory vs. error-correction types across blocks. FIG. 12A is a plot that illustrates the evolution of the empirically estimated mean shape in each block. FIG. 12B is a plot that illustrates the evolution of the empirically estimated variance of shape values. FIG. 12C is a plot that illustrates the evolution of the MVR (mean/var ratio) for the exploratory and error-correction subgroups.

FIG. 12D is a shape-scale plot that illustrates block by block evolution of the empirically estimated shape and scale parameters. Block number is proportional to the marker size, with earlier blocks having smaller size and later blocks increasing in size. Thus, the trajectory on the shape-scale plane confirms the departure from a memoryless random state (e.g., when the shape value is 1). To better visualize these processes, we zoom in and unfold the two types of learning of FIG. 12D. FIG. 12E focuses on the exploratory process. As time progresses, indicated by the arrow, the learning generally evolves from memoryless (shape 1) towards skewed, heavy tailed distributions and more symmetric distributions of the shape. FIG. 12F focuses on the error correction process. Here is seen the opposite trend whereby initially the distributions have symmetric shape (in the Gaussian range of the Γ family) but as time progresses, indicated by the arrow, the distribution shape values approach values closer to those observed for the exploratory process: skewed, heavy tailed distributions. The exploratory learning is confined to the shape values close to the memoryless exponential distribution, while the error-corrective mode evolves from higher to lower values of the Gaussian regime of the Γ family. This convergent global behavior is congruent with the convergent local behavior depicted in FIG. 9C.

Notice here that we are capturing the distribution of the fluctuations in the estimated shape parameter with a Γ process as well. We are referring to the shape and scale parameters values of the distributions derived (globally, a posteriori) from the fluctuations in shape of the MMS derived from the EEG hub channels window by window. On this shape-scale plane, the dispersion (scale of the fluctuations in shape values of MMS over all the windows in a block) along the y-axis, is larger as learning occurs, broadening the width of distribution shape values as learning takes place.

The switch from exponential to heavy tailed to Gaussian distributions reflects the more systematic confirmation of a regularity in the stimuli. Initially, all future stimuli are equally probable (exponential regime), but in time, correct prediction of futures events increases, consistent with the transition from a detected regularity to a systematic goal. Once a goal is in place, error correction is the learning regime reflecting Gaussian predictive process. Here is where we see a tendency to symmetric shapes approached by both modes along the shape axis of the shape-scale plane. One mode (the exploratory) approaching it from the left, away from the memoryless exponential. The other approaching it from the right.

The stochastic transition depicted in FIG. 12E and FIG. 12F confirms the separation between two fundamentally different learning styles with initially different stochastic regimes. It also highlights a phase transition approximately midway of the learning progression. Notwithstanding the initial differences, these regimes converged to similar signatures in the end. This transition from memoryless exploration (exponential) to predictive error-correction (heavy-tailed to Gaussian) emerges midway of the session, blocks 3 to 4.

Likely the regularity then self-emerges and eventually, through guess and systematic confirmation, transitions to a steady goal, one that serves as a standard from which to compute an error. In FIG. 12E and FIG. 12F is seen the system transitioning from an initial purposeless search to a search that then acquires a clear purpose, i.e., self-discovery of a task goal that was not instructed to the system.

Our results suggest that this transition from memoryless into error correction-based learning depends on some minimum level of explicit knowledge. Examining this global process, it appears that one subgroup (type B) had enough explicit knowledge to trigger this transition much earlier than in the other subgroup. The subgroup (type A) employing an initial exploratory mode, for which the search was in the here and now, did not acquire distributions of the shape parameter away from the exponential range until around blocks 3-4. This was when the system shifted to a Gaussian mode (FIG. 12E larger markers) and when locally the variance of the MMS shrunk (FIG. 9A and FIG. 9B), thus raising the MVR of the fluctuations in shape parameter on the global time frame of the entire learning session (FIG. 11C). In this exploratory regime, the system does not immediately progress into acquiring a predictive code. In other words, because of not yet committing to regularities in the perceptual input, the predictive processing that underwrites exploitative or goal-directed behavior is initially precluded in favor of broadening the acceptance of information that enables surprise and self-referencing towards the self-discovery of a goal. Only then, does the system transition into an error-corrective regime.

We can appreciate that the mixed case yields the most toward-Gaussian-predictive shifts in distribution change, with the highest shape value. This is accompanied by the highest MVR (i.e., at the lowest Γ scale value.)

At a global timescale (e.g., stochastic trajectory of the empirically estimated parameters examined a posteriori, across the entire experimental learning session) we assessed the change in stochastic variations of the signals over time. To do so, we examined the evolution of the fluctuations in the change of shape values using the Earth Movers Distance (EMD) metric. We compared from trial to trial, and block to block, across participants, the fluctuations in the amplitude of the change in distributions of the shape parameter values as measured by the EMD. We also assessed the rate of the change in peaks (inter peak intervals related to the physical timing of the overall global process by our unit of time, 5-second windows with 50% overlap.) These parameters are analogous to a kinematic “speed temporal profile” of the pdfs' shape trajectory. As the distribution employed by a subject shifts stochastic signatures per unit time on the shape-scale plane, we obtain enough to estimate the shape and scale parameter values of each window with tight 95% confidence intervals. The EMD scalar profile over time, measuring how the histograms used in the estimation process change from window to window, reflect the dynamic nature of the stochastic shifts that occur as the participants perform the task and learn in exploratory, or in error-correction mode, converging toward the signatures of the latter at the end of the learning process.

The analyses revealed that the system clearly distinguishes the rates at which the distributions change shape from the random to the correlated groups and between those and the mixed group.

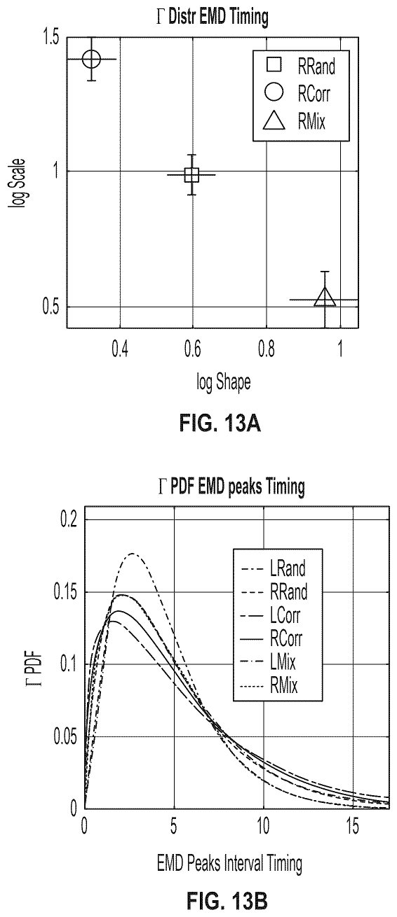

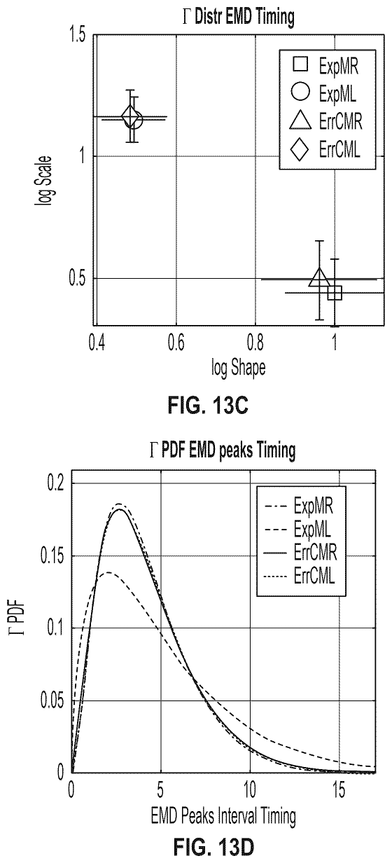

FIG. 13A through FIG. 13D are plots that illustrate distributions of distances between successive micropeak distributions during experimental learning sessions by one or more subjects, according to an embodiment. FIG. 13A shows this on the log-log shape-scale plane where each point with 95% confidence intervals, represents the performance for the right target (left not shown for simplicity but has similar patterns). The corresponding pdfs for both right and left oriented targets are shown in FIG. 13B. FIG. 13C shows on a log-log shape-scale plane the differentiation between targets for the two subgroups of the mixed stimuli at the global level. The corresponding pdfs are shown in FIG. 13D.

These rates of change in the two subgroups of the mixed case, clearly distinguish the left from the right oriented targets, with comparable rates of shifts in distribution shape values for the exploratory and the error-correction types. These are shown in FIG. 13C (shape and scale parameters values fit to EMD peak intervals data) and 13D (corresponding pdfs.) These transitions reveal similar rates of change in the interpeak intervals, suggesting smooth transitions in both exploratory and error corrective cases. These comparable shifts in distribution dynamics for exploratory and error correction stochastic regimes hint at a smooth process whether the system is curiously wondering in exploratory mode, or aiming for a task goal, in error-correction mode.

### 2.2 Experiments During Dance Task Learning

A similar experiment was performed for a male subject learning dance moves from a female partner or an avatar. In this experiment heart signals, movement and EEG data were obtained, and processed as described with reference to FIG. 6. The heart signal is shifted in real time in the male upon ensonifying the female's traces and blending it with music. Furthermore, the learning to interact with an avatar on the screen is also used where the timing of the projected avatar movie is shifted. Micropeaks in the subject's sensors and video are tracked while the subject learns to play catch up in real time. In some embodiments, regions of interest were embedded across space where the subject is allowed to move so that the subject triggers music with the subject's body movement. The subject's micropeaks were tracked as the subject traversed the space and self-discovered which body part caused this music triggering effect.

### 2.3 Experiments for ASD Detection

Using video stimulus and video camera data, and the methods described above, micropeaks were tracked for a population of ASD subjects and a population of typically developing (TD) subjects. Both populations manifest the presence of the memoryless exponential distribution accompanied by the high EMD signal and low variance (exploratory learning). However, the ASD children switch faster to error-correction learning, while the TD have a different profile of error-correction learning than the ASD. The TD population continues to learn in error correction and shrink the variance towards the very end of the learning session. While the ASD children attain low variance (error-correction) earlier in the learning session, the variance plateaus and never really rises or falls substantially. Thus, the TD population surpasses the ASD in ultimately attaining lower variance. In this sense, it is possible to
1) detect the transition point in time, which differs between the two (faster in ASD) and
2) track the error correction learning which is different in ASD vs TD.

So, the differentiation between the two populations is in the unfolding of these learning types.

### 3. COMPUTATIONAL HARDWARE OVERVIEW

FIG. 14 is a block diagram that illustrates a computer system 1400 upon which an embodiment of the invention may be implemented. Computer system 1400 includes a communication mechanism such as a bus 1410 for passing information between other internal and external components of the computer system 1400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1400, or a portion thereof, constitutes a means for performing one or more steps of one or more methods described herein.

A sequence of binary digits constitutes digital data that is used to represent a number or code for a character. A bus 1410 includes many parallel conductors of information so that information is transferred quickly among devices coupled to the bus 1410. One or more processors 1402 for processing information are coupled with the bus 1410. A processor 1402 performs a set of operations on information. The set of operations include bringing information in from the bus 1410 and placing information on the bus 1410. The set of operations also typically include comparing two or more units of information, shifting positions of units of information, and combining two or more units of information, such as by addition or multiplication. A sequence of operations to be executed by the processor 1402 constitutes computer instructions.

Computer system 1400 also includes a memory 1404 coupled to bus 1410. The memory 1404, such as a random access memory (RAM) or other dynamic storage device, stores information including computer instructions. Dynamic memory allows information stored therein to be changed by the computer system 1400. RAM allows a unit of information stored at a location called a memory address to be stored and retrieved independently of information at neighboring addresses. The memory 1404 is also used by the processor 1402 to store temporary values during execution of computer instructions. The computer system 1400 also includes a read only memory (ROM) 1406 or other static storage device coupled to the bus 1410 for storing static information, including instructions, that is not changed by the computer system 1400. Also coupled to bus 1410 is a non-volatile (persistent) storage device 1408, such as a magnetic disk or optical disk, for storing information, including instructions, that persists even when the computer system 1400 is turned off or otherwise loses power.

Information, including instructions, is provided to the bus 1410 for use by the processor from an external input device 1412, such as a keyboard containing alphanumeric keys operated by a human user, or a sensor. A sensor detects conditions in its vicinity and transforms those detections into signals compatible with the signals used to represent information in computer system 1400. Other external devices coupled to bus 1410, used primarily for interacting with humans, include a display device 1414, such as a cathode ray tube (CRT) or a liquid crystal display (LCD), for presenting images, and a pointing device 1416, such as a mouse or a trackball or cursor direction keys, for controlling a position of a small cursor image presented on the display 1414 and issuing commands associated with graphical elements presented on the display 1414.

In the illustrated embodiment, special purpose hardware, such as an application specific integrated circuit (IC) 1420, is coupled to bus 1410. The special purpose hardware is configured to perform operations not performed by processor 1402 quickly enough for special purposes. Examples of application specific ICs include graphics accelerator cards for generating images for display 1414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1400 also includes one or more instances of a communications interface 1470 coupled to bus 1410. Communication interface 1470 provides a two-way communication coupling to a variety of external devices that operate with their own processors, such as printers, scanners and external disks. In general the coupling is with a network link 1478 that is connected to a local network 1480 to which a variety of external devices with their own processors are connected. For example, communication interface 1470 may be a parallel port or a serial port or a universal serial bus (USB) port on a personal computer. In some embodiments, communications interface 1470 is an integrated services digital network (ISDN) card or a digital subscriber line (DSL) card or a telephone modem that provides an information communication connection to a corresponding type of telephone line. In some embodiments, a communication interface 1470 is a cable modem that converts signals on bus 1410 into signals for a communication connection over a coaxial cable or into optical signals for a communication connection over a fiber optic cable. As another example, communications interface 1470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1470 sends and receives electrical, acoustic or electromagnetic signals, including infrared and optical signals, that carry information streams, such as digital data.

The term computer-readable medium is used herein to refer to any medium that participates in providing information to processor 1402, including instructions for execution. Such a medium may take many forms, including, but not limited to, non-volatile media, volatile media and transmission media. Non-volatile media include, for example, optical or magnetic disks, such as storage device 1408. Volatile media include, for example, dynamic memory 1404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1402, except for carrier waves and other signals.

Logic encoded in one or more tangible media includes one or both of processor instructions on a computer-readable storage media and special purpose hardware, such as ASIC*1420.

Network link 1478 typically provides information communication through one or more networks to other devices that use or process the information. For example, network link 1478 may provide a connection through local network 1480 to a host computer 1482 or to equipment 1484 operated by an Internet Service Provider (ISP). ISP equipment 1484 in turn provides data communication services through the public, world-wide packet-switching communication network of networks now commonly referred to as the Internet 1490. A computer called a server 1492 connected to the Internet provides a service in response to information received over the Internet. For example, server 1492 provides information representing video data for presentation at display 1414.

The invention is related to the use of computer system 1400 for implementing the techniques described herein. According to one embodiment of the invention, those techniques are performed by computer system 1400 in response to processor 1402 executing one or more sequences of one or more instructions contained in memory 1404. Such instructions, also called software and program code, may be read into memory 1404 from another computer-readable medium such as storage device 1408. Execution of the sequences of instructions contained in memory 1404 causes processor 1402 to perform the method steps described herein. In alternative embodiments, hardware, such as application specific integrated circuit 1420, may be used in place of or in combination with software to implement the invention. Thus, embodiments of the invention are not limited to any specific combination of hardware and software.

The signals transmitted over network link 1478 and other networks through communications interface 1470, carry information to and from computer system 1400. Computer system 1400 can send and receive information, including program code, through the networks 1480, 1490 among others, through network link 1478 and communications interface 1470. In an example using the Internet 1490, a server 1492 transmits program code for a particular application, requested by a message sent from computer 1400, through Internet 1490, ISP equipment 1484, local network 1480 and communications interface 1470. The received code may be executed by processor 1402 as it is received, or may be stored in storage device 1408 or other non-volatile storage for later execution, or both. In this manner, computer system 1400 may obtain application program code in the form of a signal on a carrier wave.

Various forms of computer readable media may be involved in carrying one or more sequence of instructions or data or both to processor 1402 for execution. For example, instructions and data may initially be carried on a magnetic disk of a remote computer such as host 1482. The remote computer loads the instructions and data into its dynamic memory and sends the instructions and data over a telephone line using a modem. A modem local to the computer system 1400 receives the instructions and data on a telephone line and uses an infra-red transmitter to convert the instructions and data to a signal on an infra-red a carrier wave serving as the network link 1478. An infrared detector serving as communications interface 1470 receives the instructions and data carried in the infrared signal and places information representing the instructions and data onto bus 1410. Bus 1410 carries the information to memory 1404 from which processor 1402 retrieves and executes the instructions using some of the data sent with the instructions. The instructions and data received in memory 1404 may optionally be stored on storage device 1408, either before or after execution by the processor 1402.

FIG. 15 illustrates a chip set 1500 upon which an embodiment of the invention may be implemented. Chip set 1500 is programmed to perform one or more steps of a method described herein and includes, for instance, the processor and memory components described with respect to FIG.*14 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1500, or a portion thereof, constitutes a means for performing one or more steps of a method described herein.

In one embodiment, the chip set 1500 includes a communication mechanism such as a bus 1501 for passing information among the components of the chip set 1500. A processor 1503 has connectivity to the bus 1501 to execute instructions and process information stored in, for example, a memory 1505. The processor 1503 may include one or more processing cores with each core configured to perform independently. A multi-core processor enables multiprocessing within a single physical package. Examples of a multi-core processor include two, four, eight, or greater numbers of processing cores. Alternatively or in addition, the processor 1503 may include one or more microprocessors configured in tandem via the bus 1501 to enable independent execution of instructions, pipelining, and multithreading. The processor 1503 may also be accompanied with one or more specialized components to perform certain processing functions and tasks such as one or more digital signal processors (DSP) 1507, or one or more application-specific integrated circuits (ASIC) 1509. A DSP 1507 typically is configured to process real-world signals (e.g., sound) in real time independently of the processor 1503. Similarly, an ASIC 1509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1503 and accompanying components have connectivity to the memory 1505 via the bus 1501. The memory 1505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1505 also stores the data associated with or generated by the execution of one or more steps of the methods described herein.

FIG. 16 is a diagram of exemplary components of a mobile terminal 1600 (e.g., cell phone handset) for communications, which is capable of operating in the system, according to one embodiment. In some embodiments, mobile terminal 1601, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory (ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1603, a Digital Signal Processor (DSP) 1605, and a receiver/transmitter unit including a microphone gain control unit and a speaker gain control unit. A main display unit 1607 provides a display to the user in support of various applications and mobile terminal functions that perform or support the steps as described herein. The display 1607 includes display circuitry configured to display at least a portion of a user interface of the mobile terminal (e.g., mobile telephone). Additionally, the display 1607 and display circuitry are configured to facilitate user control of at least some functions of the mobile terminal. An audio function circuitry 1609 includes a microphone 1611 and microphone amplifier that amplifies the speech signal output from the microphone 1611. The amplified speech signal output from the microphone 1611 is fed to a coder/decoder (CODEC) 1613.

A radio section 1615 amplifies power and converts frequency in order to communicate with a base station, which is included in a mobile communication system, via antenna 1617. The power amplifier (PA) 1619 and the transmitter/modulation circuitry are operationally responsive to the MCU 1603, with an output from the PA 1619 coupled to the duplexer 1621 or circulator or antenna switch, as known in the art. The PA 1619 also couples to a battery interface and power control unit 1620.

In use, a user of mobile terminal 1601 speaks into the microphone 1611 and his or her voice along with any detected background noise is converted into an analog voltage. The analog voltage is then converted into a digital signal through the Analog to Digital Converter (ADC) 1623. The control unit 1603 routes the digital signal into the DSP 1605 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1625 for compensation of any frequency-dependent impairments that occur during transmission though the air such as phase and amplitude distortion. After equalizing the bit stream, the modulator 1627 combines the signal with a RF signal generated in the RF interface 1629. The modulator 1627 generates a sine wave by way of frequency or phase modulation. In order to prepare the signal for transmission, an up-converter 1631 combines the sine wave output from the modulator 1627 with another sine wave generated by a synthesizer 1633 to achieve the desired frequency of transmission. The signal is then sent through a PA 1619 to increase the signal to an appropriate power level. In practical systems, the PA 1619 acts as a variable gain amplifier whose gain is controlled by the DSP 1605 from information received from a network base station. The signal is then filtered within the duplexer 1621 and optionally sent to an antenna coupler 1635 to match impedances to provide maximum power transfer. Finally, the signal is transmitted via antenna 1617 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1601 are received via antenna 1617 and immediately amplified by a low noise amplifier (LNA) 1637. A down-converter 1639 lowers the carrier frequency while the demodulator 1641 strips away the RF leaving only a digital bit stream. The signal then goes through the equalizer 1625 and is processed by the DSP 1605. A Digital to Analog Converter (DAC) 1643 converts the signal and the resulting output is transmitted to the user through the speaker 1645, all under control of a Main Control Unit (MCU) 1603 which can be implemented as a Central Processing Unit (CPU) (not shown).

The MCU 1603 receives various signals including input signals from the keyboard 1647. The keyboard 1647 and/or the MCU 1603 in combination with other user input components (e.g., the microphone 1611) comprise a user interface circuitry for managing user input. The MCU 1603 runs a user interface software to facilitate user control of at least some functions of the mobile terminal 1601 as described herein. The MCU 1603 also delivers a display command and a switch command to the display 1607 and to the speech output switching controller, respectively. Further, the MCU 1603 exchanges information with the DSP 1605 and can access an optionally incorporated SIM card 1649 and a memory 1651. In addition, the MCU 1603 executes various control functions required of the terminal. The DSP 1605 may, depending upon the implementation, perform any of a variety of conventional digital processing functions on the voice signals. Additionally, DSP 1605 determines the background noise level of the local environment from the signals detected by microphone 1611 and sets the gain of microphone 1611 to a level selected to compensate for the natural tendency of the user of the mobile terminal 1601.

The CODEC 1613 includes the ADC 1623 and DAC 1643. The memory 1651 stores various data including call incoming tone data and is capable of storing other data including music data received via, e.g., the global Internet. The software module could reside in RAM memory, flash memory, registers, or any other form of writable storage medium known in the art. The memory device 1651 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1649 carries, for instance, important information, such as the cellular phone number, the carrier supplying service, subscription details, and security information. The SIM card 1649 serves primarily to identify the mobile terminal 1601 on a radio network. The card 1649 also contains a memory for storing a personal telephone number registry, text messages, and user specific mobile terminal settings.

In some embodiments, the mobile terminal 1601 includes a digital camera comprising an array of optical detectors, such as charge coupled device (CCD) array 1665. The output of the array is image data that is transferred to the MCU for further processing or storage in the memory 1651 or both. In the illustrated embodiment, the light impinges on the optical array through a lens 1663, such as a pin-hole lens or a material lens made of an optical grade glass or plastic material. In the illustrated embodiment, the mobile terminal 1601 includes a light source 1661, such as a LED to illuminate a subject for capture by the optical array, e.g., CCD 1665. The light source is powered by the battery interface and power control module 1620 and controlled by the MCU 1603 based on instructions stored or loaded into the MCU 1603.

### 4. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 5. REFERENCES

1. [1] N. Censor, D. Sagi, and L. G. Cohen, Common mechanisms of human perceptual and motor learning. Nat Rev Neurosci 13 (2012) 658-64.
2. [2] R. Frost, B. C. A., and M. H. C., Statistical learning research: A critical review and possible new directions. Psychological Bulletin 145 (2019) 1128.
3. [3] U. Hasson, The neurobiology of uncertainty: implications for statistical learning. Philos Trans R Soc Lond B Biol Sci 372 (2017).
4. [4] D. Drai, and I. Golani, SEE: a tool for the visualization and analysis of rodent exploratory behavior. Neurosci Biobehav Rev 25 (2001) 409-26.
5. [5] T. Frostig, H. Alonim, G. Scheingesicht, Y. Benjamini, and I. Golani, Exploration in the Presence of Mother in Typically and Non-typically Developing Pre-walking Human Infants. Front Behav Neurosci 14 (2020) 580972.
6. [6] P. Dayan, and B. W. Balleine, Reward, motivation, and reinforcement learning. Neuron 36 (2002) 285-98.
7. [7] R. S. Sutton, Reinforcement learning, Kluwer Academic Publishers, Boston, 1992.
8. [8] R. Dubey, and T. L. Griffiths, Understanding exploration in humans and machines by formalizing the function of curiosity. Current Opinion in Behavioral Sciences. 35 (2020) 118-124.
9. [9] C. Lanczos, The variational principles of mechanics, Dover Publications, New York, 1986.
10. [10] N. A. Bernstein, The Coordination and Regulation of Movements, Pergamon Press, London, 1967.
11. [11] E. B. Torres, Zipser, D., Reaching to Grasp with a Multi-jointed Arm (I): A Computational Model. Journal of Neurophysiology 88 (2002) 1-13.
12. [12] K. Friston, T. FitzGerald, F. Rigoli, P. Schwartenbeck, O. D. J, and G. Pezzulo, Active inference and learning. Neurosci Biobehav Rev 68 (2016) 862-879.
13. [13] K. J. Friston, M. Lin, C. D. Frith, G. Pezzulo, J. A. Hobson, and S. Ondobaka, Active Inference, Curiosity and Insight. Neural Comput 29 (2017) 2633-2683.
14. [14] C. M. Conway, How does the brain learn environmental structure? Ten core principles for understanding the neurocognitive mechanisms of statistical learning. Neurosci Biobehav Rev. 112 (2020) 279-299.
15. [15] M. H. Christiansen, Implicit Statistical Learning: A Tale of Two Literatures. Top Cogn Sci 11 (2019) 468-481.
16. [16] A. R. Romberg, and J. R. Saffran, Statistical learning and language acquisition. Wiley Interdiscip Rev Cogn Sci 1 (2010) 906-914.
17. [17] J. R. Saffran, R. N. Aslin, and E. L. Newport, Statistical learning by 8-month-old infants. Science 274 (1996) 1926-8.
18. [18] P. Sinha, Autism as a disorder of prediction. Proc Natl Acad Sci USA 42 (2014) 15220-15225.
19. [19] E. B. Torres, M. Brincker, R. W. Isenhower, P. Yanovich, K. A. Stigler, J. I. Nurnberger, D. N. Metaxas, and J. V. Jose, Autism: the micro-movement perspective. Front Integr Neurosci 7 (2013) 32.
20 [20] C. Crivello, S. Phillips, and D. Poulin-Dubois, Selective social learning in infancy: looking for mechanisms. Developmental science 21 (2018) e12592.
21. [21] H. Schwarb, and E. H. Schumacher, Generalized lessons about sequence learning from the study of the serial reaction time task. Advances in cognitive psychology 8 (2012) 165.
22. [22] M. A. Kunar, F. S., H. T. S., and J. M. Wolfe, Does contextual cuing guide the deployment of attention? Journal of Experimental Psychology: Human Perception and Performance 33 (2007) 816.
23. [23] A. Vaskevich, A. Nishry, Y. Smilansky, and R. Luria, Neural Evidence Suggests Both Interference and Facilitation from Embedding Regularity into Visual Search. J Cogn Neurosci 33 (2021) 622-634.
24. [24] E. B. Torres, Two classes of movements in motor control. Exp Brain Res 215 (2011) 269-83.
25. [25] E. B. Torres, B. Smith, S. Mistry, M. Brincker, and C. Whyatt, Neonatal Diagnostics: Toward Dynamic Growth Charts of Neuromotor Control. Front Pediatr 4 (2016) 121.
26. [26] J. Ryu, T. Bar-Shalita, Y. Granovsky, I. Weissman-Fogel, and E. B. Torres, Personalized Biometrics of Physical Pain Agree with Psychophysics by Participants with Sensory over Responsivity. J Pers Med 11 (2021).
27. [27] N. Bigdely-Shamlo, T. Mullen, C. Kothe, K. M. Su, and K. A. Robbins, The PREP pipeline: standardized preprocessing for large-scale EEG analysis. Front Neuroinform 9 (2015) 16.
28. [28] A. Phinyomark, S. Thongpanja, H. Hu, P. Phukpattaranont, and C. Limsakul, The usefulness of mean and median frequencies in electromyography analysis. Computational intelligence in electromyography analysis-A perspective on current applications and future challenges. (2012) 195-220.
29. [29] E. B. Torres, Methods for the diagnosis and treatment of neurological disorders. in: USPTO, (Ed.), Google Patents, Rutgers State University of New Jersey, US, 2018, pp. 38.
30. [30] J. Lleonart, Salat, J., and Torres, G. J., Removing allometric effects of body size in morphological analysis. J. Theor. Biol. 205 (2000) 85-93.
31. [31] G. Monge, Memoire sur la theorie des deblais et des remblais., Histoire de l′ Academie Royale des Science; avec les Memoires de Mathematique et de Physique;, De L′imprimerie Royale, Paris, France, 1781.
32. [32] Y. Rubner, C. Tomasi, and L. J. Guibas, Metric for Distributions with Applications to Image Databases., Proceedings of the ICCV, Bombay, India, 1998.
33. [33] A. Vaskevich, and R. Luria, Adding statistical regularity results in a global slowdown in visual search. Cognition 174 (2018) 19-27.
34. [34] J. A. Hartigan, and P. M. Hartigan, The Dip Test of Unimodality. The Annals of Statistics 13 (1985) 70-84.
35. [35] E. B. Torres, and B. Lande, Objective and personalized longitudinal assessment of a pregnant patient with post severe brain trauma. Front Hum Neurosci 9 (2015) 128.
36. [36] S. Nastase, V. Iacovella, and U. Hasson, Uncertainty in visual and auditory series is coded by modality-general and modality-specific neural systems. Hum Brain Mapp 35 (2014) 1111-28.
37 [37] M. Zellin, M. Conci, A. von Mühlenen, and H. J. Müller, Here today, gone tomorrow-adaptation to change in memory-guided visual search. PloS one 8 (2013) e59466.
38. [38] T. Makovski, and Y. V. Jiang, Contextual cost: When a visual-search target is not where it should be. Quarterly Journal of Experimental Psychology 63 (2010) 216-225.
39. [39] A. Vaskevich, and R. Luria, Statistical learning in visual search is easier after experience with noise than overcoming previous learning. Visual Cognition 27 (2019) 537-550.
40. [40] L. J. Batterink, K. A. Paller, and P. J. Reber, Understanding the Neural Bases of Implicit and Statistical Learning. Top Cogn Sci 11 (2019) 482-503.
41. [41] J. Moser, L. Batterink, Y. Li Hegner, F. Schleger, C. Braun, K. A. Paller, and H. Preissl, Dynamics of nonlinguistic statistical learning: From neural entrainment to the emergence of explicit knowledge. Neuroimage 240 (2021) 118378.
42. [42] B. Toth, K. Janacsek, A. Takacs, A. Kobor, Z. Zavecz, and D. Nemeth, Dynamics of EEG functional connectivity during statistical learning. Neurobiol Learn Mem 144 (2017) 216-229.
43. [43] L. Bogaerts, C. G. Richter, A. N. Landau, and R. Frost, Beta-Band Activity Is a Signature of Statistical Learning. J Neurosci 40 (2020) 7523-7530.
44. [44] R. Dale, N. Duran, and R. Morehead, Prediction during statistical learning, and implications for the implicit/explicit divide. Adv. Cogn. Psychol. 8 (2012) 196-209.
45. [45] K. Nishikawa, S. Murray, and M. Flanders, Do arm postures vary with the speed of reaching? Journal of Neurophysiology 81 (1999) 2582-6.
46. [46] C. G. Atkeson, and Hollerbach, J. M, Kinematics Features of unrestrained vertical arm movements. Journal of Neuroscience 5 (1985) 2318-2330.
47. [47] E. B. Torres, Zipser D., Simultaneous control of hand displacements and rotations in orientation-matching experiments. Journal of Applied Physiology 96 (2004) 1978-1987.
48. [48] E. Torres, and R. Andersen, Space-time separation during obstacle-avoidance learning in monkeys. J Neurophysiol 96 (2006) 2613-32.
49. [49] C. R. Gallistel, and J. Gibbon, Time, rate, and conditioning. Psychol Rev 107 (2000) 289-344.
50. [50] M. Brincker, and E. B. Torres, Chapter 1-Why Study Movement Variability in Autism_. in: E. B. Torres, and C. Whyatt, (Eds.), Autism: the movement sensing perspective, CRC Press/Taylor & Francis Group, Boca Raton, 2018, pp. xviii, 386 pages.
51. [51] S. Grillner, and A. El Manira, Current Principles of Motor Control, with Special Reference to Vertebrate Locomotion. Physiol Rev 100 (2020) 271-320.
52. [52] E. Thelen, (Ed.), Mechanisms of Cognitive Development: Behavioral and Neural Perspectives Lawrence Erlbaum Associates Inc., Pittsburgh, PA, 2001.
53. [53] D. Drai, Y. Benjamini, and I. Golani, Statistical discrimination of natural modes of motion in rat exploratory behavior. J Neurosci Methods 96 (2000) 119-31.
54. [54] C. Kidd, S. T. Piantadosi, and R. N. Aslin, The Goldilocks effect: human infants allocate attention to visual sequences that are neither too simple nor too complex. PLOS One 7 (2012) e36399.
55. [55] H. L. More, and J. M. Donelan, Scaling of sensorimotor delays in terrestrial mammals. Proc Biol Sci. 285 (2018) 1885.
56. [56] J. W. Krakauer, P. Mazzoni, A. Ghazizadeh, R. Ravindran, and R. Shadmehr, Generalization of motor learning depends on the history of prior action. PLOS Biol 4 (2006) e316.
57. [57] H. Tanaka, and T. J. Sejnowski, Motor adaptation and generalization of reaching movements using motor primitives based on spatial coordinates. J Neurophysiol 113 (2015) 1217-33.
58. [58] H. G. Wu, and M. A. Smith, The generalization of visuomotor learning to untrained movements and movement sequences based on movement vector and goal location remapping. J Neurosci 33 (2013) 10772-89.
59. [59] E. B. Torres, P. Yanovich, and D. N. Metaxas, Give spontaneity and self-discovery a chance in ASD: spontaneous peripheral limb variability as a proxy to evoke centrally driven intentional acts. Front Integr Neurosci 7 (2013) 46.
60. [60] D. Pathak, P. Agrawal, A. A. Efros, and T. Darrell, Curiosity-driven Exploration by Self-supervised Prediction. in: Y. W. T. D. Precup, (Ed.), Proceedings of the 34th International Conference on Machine Learning, MLResearch Press, Sydney, Australia, 2017, pp. 2778-2787.
61. [61] N. D. Daw, J. P. O'Doherty, P. Dayan, B. Seymour, and R. J. Dolan, Cortical substrates for exploratory decisions in humans. Nature 441 (2006) 876-9.
62. [62] D. Y. Little, and F. T. Sommer, Learning and exploration in action-perception loops. Front Neural Circuits 7 (2013) 37.
63. [63] S. Still, and D. Precup, An information-theoretic approach to curiosity-driven reinforcement learning. Theory Biosci 131 (2012) 139-48.
64. [64] P. Schwartenbeck, J. Passecker, T. U. Hauser, T. H. FitzGerald, M. Kronbichler, and K. J. Friston, Computational mechanisms of curiosity and goal-directed exploration. Elife 8 (2019).
65. [65] A. Baranes, and P. Y. Oudeyer, Robust Intrinsically Motivated Exploration and Active Learning. Ieee Transactions on Autonomous Mental Development 1 (2009) 155-169.
66 [66] J. Schmidhuber, Formal Theory of Creativity, Fun, and Intrinsic Motivation (1990-2010). Ieee Transactions on Autonomous Mental Development 2 (2010) 230-247.
67. [67] K. Friston, J. Mattout, and J. Kilner, Action understanding and active inference. Biol Cybern 104 (2011) 137-60.
68. [68] J. Winn, and C. M. Bishop, Variational message passing. Journal of Machine Learning Research 6 (2005) 661-694.
69 [69] T. Parr, and K. J. Friston, Generalised free energy and active inference. Biol Cybern 113 (2019) 495-513.
70 [70] J. O. Berger, Statistical decision theory and Bayesian analysis, Springer-Verlag, New York, 1993.
71. [71] D. J. C. Mackay, Information theory, inference, and learning algorithms, Cambridge University Press, Cambridge, UK; New York, 2003.
72. [72] E. B. Torres, Objective biometric methods for the diagnosis and treatment of nervous system disorders, Academic Press, London, 2018.

What is claimed is:

1. A method implemented on a processor for distinguishing exploratory learning from error-correction learning in a subject, the method comprising:
presenting, to a subject, sensory stimuli beginning at a start time and ending at an end time, wherein a stimuli time difference between the start time and the end time is more than 10 minutes and less than 12 hours and is divided into a plurality of time blocks of equal duration;
determining Gamma function (Γ) scale and shape parameter value pair for micropeak statistics within sensor data observing said subject during each window of one or more windows of equal duration in each time block of the plurality of time blocks, wherein each window has a duration such that an average number of micropeaks in the windows in the block is at least 100 micropeaks;
determining a profile of Γ scale and shape parameter value pairs with time during each block; and
determining that the subject is engaged in exploratory learning during each block in which variance in that block exceeds a threshold variance based on a Γ scale and shape parameter value pair selected along the profile.

2. The method as recited in claim 1, wherein the sensory stimuli includes predictable and unpredictable changes.

3. The method as recited in claim 1, wherein the sensory stimuli comprises visual-motor stimuli.

4. The method as recited in claim 1, wherein the sensor data is collected at a rate greater than 100 Hertz (Hz), or greater than 150 Hz, or greater than 200 Hz, or greater than 250 Hertz, or greater than 300 Hz.

5. The method as recited in claim 1, wherein each window has a duration selected in a range from one second to 10 seconds.

6. The method as recited in claim 1, wherein each window overlaps in time any adjacent window.

7. The method as recited in claim 1, wherein each window overlaps any adjacent window in time by 50% of the window duration.

8. The method as recited in claim 1, wherein each block includes at least 20 windows.

9. The method as recited in claim 1, further comprising determining distance between distributions in successive windows wherein distance is Earth Mover's distance (EMD).

10. The method as recited in claim 1, wherein the threshold is a percentile of Γ scale values along the profile.

11. The method as recited in claim 10, wherein the percentile is the 50th percentile.

12. The method as recited in claim 1, further comprising determining a neurological condition of the subject based upon a percentage of time during the presentation of the sensory stimuli during which the subject is engaged in exploratory learning.

13. A non-transitory computer-readable medium carrying one or more sequences of instructions for distinguishing exploratory learning from error-correction learning in a subject, wherein execution of the one or more sequences of instructions by one or more processors causes the one or more processors to:
present on a display device viewable by a subject, sensory stimuli beginning at a start time and ending at an end time, wherein a stimuli time difference between the start time and the end time is more than 10 minutes and less than 12 hours and is divided into a plurality of time blocks of equal duration;
determine Gamma function (Γ) scale and shape parameter value pair for micropeak statistics within sensor data observing said subject during each window of one or more windows of equal duration in each time block of the plurality of time blocks, wherein each window has a duration such that an average number of micropeaks in the windows in the block is at least 100 micropeaks;
store in a data structure a profile of Γ scale and shape parameter value pairs with time during each block; and
present on the display device data indicating that the subject is engaged in exploratory learning during each block in which variance in that block exceeds a threshold variance based on a Γ scale and shape parameter value pair selected along the profile.

14. The computer-readable medium as recited in claim 13, wherein the sensory stimuli includes predictable and unpredictable changes.

15. The computer-readable medium as recited in claim 13, wherein the sensory stimuli comprises visual-motor stimuli.

16. The computer-readable medium as recited in claim 13, wherein the sensor data is collected at a rate greater than 100 Hertz (Hz), or greater than 150 Hz, or greater than 200 Hz, or greater than 250 Hertz, or greater than 300 Hz.

17. The computer-readable medium as recited in claim 13, wherein each window has a duration selected in a range from one second to 10 seconds.

18. The computer-readable medium as recited in claim 13, wherein each window overlaps in time any adjacent window.

19. The computer-readable medium as recited in claim 13, wherein each window overlaps any adjacent window in time by 50% of the window duration.

20. The computer-readable medium as recited in claim 13, wherein each block includes at least 20 windows.

21. The computer-readable medium as recited in claim 13, wherein execution of the one or more sequences of instructions by the one or more processors further causes the one or more processors to determine distance between distributions in successive windows, wherein distance is Earth Mover's distance (EMD).

22. The computer-readable medium as recited in claim 13, wherein the threshold is a percentile of Γ scale values along the profile.

23. The computer-readable medium as recited in claim 22, wherein the percentile is the 50th percentile.

24. The computer-readable medium as recited in claim 13, wherein execution of the one or more sequences of instructions by the one or more processors further causes the one or more processors to present on the display device a neurological condition of the subject based upon a percentage of time during the presentation of the sensory stimuli during which the subject is engaged in exploratory learning.

25. A system for distinguishing exploratory learning from error-correction learning in a subject, said system comprising:
a sensor;
a display device;
at least one processor; and
at least one memory including one or more sequences of instructions,
the at least one memory and the one or more sequences of instructions configured to, with the at least one processor, cause the system to perform at least:
present on the display device viewable by a subject, sensory stimuli beginning at a start time and ending at an end time, wherein a stimuli time difference between the start time and the end time is more than 10 minutes and less than 12 hours and is divided into a plurality of time blocks of equal duration;
determine Gamma function (I) scale and shape parameter value pair for micropeak statistics within sensor data from the sensor observing said subject during each window of one or more windows of equal duration in each time block of the plurality of time blocks, wherein each window has a duration such that an average number of micropeaks in the windows in the block is at least 100 micropeaks;
store in a data structure a profile of Γ scale and shape parameter value pairs with time during each block; and
present on the display device data indicating that the subject is engaged in exploratory learning during each block in which variance in that block exceeds a threshold variance based on a Γ scale and shape parameter value pair selected along the profile.

26. The system as recited in claim 25, wherein the sensory stimuli includes predictable and unpredictable changes.

27. The system as recited in claim 25, wherein the sensory stimuli comprises visual-motor stimuli.

28. The system as recited in claim 25, wherein the sensor data is collected at a rate greater than 100 Hertz (Hz), or greater than 150 Hz, or greater than 200 Hz, or greater than 250 Hertz, or greater than 300 Hz.

29. The system as recited in claim 25, wherein each window has a duration selected in a range from one second to 10 seconds.

30. The system as recited in claim 25, wherein each window overlaps in time any adjacent window.

31. The system as recited in claim 25, wherein each window overlaps any adjacent window in time by 50% of the window duration.

32. The system as recited in claim 25, wherein each block includes at least 20 windows.

33. The system as recited in claim 25, wherein execution of the one or more sequences of instructions by the one or more processors further causes the one or more processors to determining distance between distributions in successive windows, wherein distance is Earth Mover's distance (EMD).

34. The system as recited in claim 25, wherein the threshold is a percentile of Γ scale values along the profile.

35. The system as recited in claim 34, wherein the percentile is the 50th percentile.

36. The system as recited in claim 25, wherein execution of the one or more sequences of instructions by the one or more processors further causes the one or more processors to present on the display device a neurological condition of the subject based upon a percentage of time during the presentation of the sensory stimuli during which the subject is engaged in exploratory learning.

37. A method implemented on a processor for adjusting values of parameters for a machine learning system, the method comprising adjusting parameter values for the machine learning system using exploratory learning, wherein exploratory learning comprises a sequence of one or more probability density functions used by a subject engaged in a block of exploratory learning determined by:
presenting, to a subject, sensory stimuli beginning at a start time and ending at an end time, wherein a stimuli time difference between the start time and the end time is more than 10 minutes and less than 12 hours and is divided into a plurality of time blocks of equal duration;
determining Gamma function (Γ) scale and shape parameter value pair that fit micropeak statistics within sensor data observing said subject during each window of one or more windows of equal duration in each time block of the plurality of time blocks, wherein each window has a duration such that an average number of micropeaks in the windows in the block is at least 100 micropeaks;
determining a profile of Γ scale and shape parameter value pairs during each block; and
determining that the subject is engaged in exploratory learning during each block in which variance in that block exceeds a threshold variance based on a Γ scale and shape parameter value pair selected along the profile.

38. A non-transitory computer-readable medium carrying one or more sequences of instructions for adjusting values of parameters for a machine learning system, wherein execution of the one or more sequences of instructions by one or more processors causes the one or more processors to adjust parameter values for the machine learning system using exploratory learning, wherein exploratory learning comprises a sequence of one or more probability density functions used by a subject engaged in a block of exploratory learning determined by:
presenting, to a subject, sensory stimuli beginning at a start time and ending at an end time, wherein a stimuli time difference between the start time and the end time is more than 10 minutes and less than 12 hours and is divided into a plurality of time blocks of equal duration;
determining Gamma function (Γ) scale and shape parameter value pair that fit micropeak statistics within sensor data observing said subject during each window of one or more windows of equal duration in each time block of the plurality of time blocks, wherein each window has a duration such that an average number of micropeaks in the windows in the block is at least 100 micropeaks;
determining a profile of Γ scale and shape parameter value pairs during each block; and
determining that the subject is engaged in exploratory learning during each block in which variance in that block exceeds a threshold variance based on a Γ scale and shape parameter value pair selected along the profile.

39. A system for adjusting values of parameters for a machine learning system, the system comprising:
at least one processor;
at least one memory including one or more sequences of instructions,
the at least one memory and the one or more sequences of instructions configured to, with the at least one processor, cause the system to perform adjusting parameter values for the machine learning system using exploratory learning, wherein exploratory learning comprises a sequence of one or more probability density functions used by a subject engaged in a block of exploratory learning determined by:
presenting, to a subject, sensory stimuli beginning at a start time and ending at an end time, wherein a stimuli time difference between the start time and the end time is more than 10 minutes and less than 12 hours and is divided into a plurality of time blocks of equal duration;
determining Gamma function (Γ) scale and shape parameter value pair that fit micropeak statistics within sensor data observing said subject during each window of one or more windows of equal duration in each time block of the plurality of time blocks, wherein each window has a duration such that an average number of micropeaks in the windows in the block is at least 100 micropeaks;
determining a profile of Γ scale and shape parameter value pairs during each block; and
determining that the subject is engaged in exploratory learning during each block in which variance in that block exceeds a threshold variance based on a Γ scale and shape parameter value pair selected along the profile.
